SEQUENCE LISTING (53 sequences)

SEQ ID NO: 1 - DNA, 3133 bp, Rattus sp.
  CDS (76)...(3036)
gcacgaggggacaagagcgggtctggctggggtgtctccttcccttcgcacagcacagtggtaacttctttcgggatggagagtgtgcggacgctgtggctcagcgtggccctggcgctggcggtggggtcccgagtggtgcgcggtcaccctcagccctgccgggttcccacgcgcgctggggcctccgtgcgcctggcggcgctcctgccccgggcgcccgccgcccgcgcccgcgtcctagctgccctggccacccctgcgccgcggctgccgcacaacctgagtctggaactggtggccgtcgcgtccccgacccgggaccccgcgtcgctagctcgaggtctgtgccaggttctggcaccgcctggcgtggtggcctctatagcctttcccgaggcgcggcccgagctgcggctactgcagttcctggcagccgccacagagaccccagtgactccgttccatctgcagctggactgggctagtcccctggagaccatactggatgtgctggtgtccctggtacgggcacatgcctgggaggacattgctctagtactctgccgtgtccgggaccctggcagcctggtgacactctggactaaccatgctagccaggctccaaagtttgtgctggacctgagccggctggacagcaggaatgacagccttcgggctggactggccctgttgggggcgctggaaggagggggaaccccagtgcctgcagcagtcctcctaggctgcagcactgcccgtgcacatgaggtcctagaggctgcaccaccgggtccccagtggttgctgggcacaccattgcccgctgaggcactgcccacgactggtctgccacctggcgtgctggcgctgggggaaaccgaacaacactctctggaagctgtcgtccacgacatggtggagcttgtggctcaggcactcagtagcatggcccttgtacacccagagcgggcactgcttccagctgtggtgaactgtgatgacctgaaaacaggcggatctgaggcaacagggcgcaccttggctcggtttctcggcaacacctcatttcagggccgaacaggggccgtgtgggtgacaggctcctctcaggtgcatgtgtctcggcatttcaaggtatggagcctgcgccgggatccgctgggtgccccagcctgggcaaccgtgggcagctggcaggatggacagctggacttccagccaggggcagccgctctccgagtcccatctccgtctggcacccaggcccgaccaaagctgcgtgtggtaaccctggtggaacacccgtttgtgttcaccagggaatctgatgaagacggacagtgcccagctgggcagctgtgtctggacccaggcaccaatgactcagccaggctggatgccctctttgctgcactggtgaatggctcagtacctcgaacgctgagaagatgctgctatggctactgcatcgacctgctggagcggctggccgaggacctggcctttgactttgagctctatattgtgggggatggcaagtacggggccctgcgtgatgggcgctggacgggcctggtgggtgacctgctggctggccgggcacacatggctgtgaccagcttcagcatcaactcagctcgctctcaggtggtggatttcaccagccctttcttctccaccagcctggggattatggtgcgcacgagagacacggcctcgcccatcggggctttcatgtggcccctgcactggtccatgtgggtgggcgtgtttgctgctctgcacctcacagcgctctttctcaccctgtacgaatggcgaagtccctacgggctcacgccgcgcggccgcaaccgtggcactgtcttctcttactcctccgcgctcaacctgtgctatgccattctctttggacgcactgtctccagtaagacgcccaagtgccctactggacgcttcctcatgaacctctgggcaatcttctgcctgctggtgctttccagttacacggccaacctggctgctgtcatggttggggacaaaacctttgaggagctgtctggaatccatgatcccaagctgcaccacccttcccaaggctttcgctttggcaccgtatgggagagcagcgcggaggcctacatcaaggcaagcttccctgagatgcacgcacacatgcgtcggcacagcgcacccaccactccacatggggtggccatgctcacgagcgacccgcccaagctcaacgccttcatcatggataaatcactactggactatgaggtgtccatagatgcggactgcaagctgctcaccgttggcaaaccctttgctatcgagggctacggcatagggctaccccaaaactcgccgctcacctccaacctgtcggagttcatcagtaggtacaagtcttcaggcttcattgatctgctccatgacaagtggtacaagatggtgccttgcgggaagcgggtgttcgccgtgacggagacgctgcagatgggggtctaccacttctcaggattgtttgtcctgctgtgcctcgggctgggcagcgcgcttctcacctctctgggtgagcatgtcttctaccgcctggtgctgccgcgcatccgcaggggtaataagctgcagtattggcttcacacgagccagaagatccaccgagccctcaatacaggaccacccgaggggcaacaggagagggcagagcaggagcgcagcggccccaaggacgagctgcctgccaccgatggtgcagggcgctggaggcgggtgcgccgggctgtggaacgggagcgacgcgtgcgtttcctgctggaacctggggaggctggcggagaccgcccgtggctctgctccaacgggcccgggctgcaagcggagctgcgggagctggagctgcgcattgaggctgcacgggagcagctgcgcagtgcgctgttgcggcgcggggagctgcgggccctgcttggggatggcacccggctcaggccactgcgcctgttgcatgcggcgcctgctgagagctgaggaaccacaaggccgcactgtccacgacagtttattctatatacaaacacgactctgtacactgcaattaaatagcgtggaacgtgaaaaaaaa

SEQ ID NO: 2 - protein, 987 aa, Rattus sp.
MESVRTLWLSVALALAVGSRVVRGHPQPCRVPTRAGASVRLAALLPRAPAARARVLAALATPAPRLPHNLSLELVAVASPTRDPASLARGLCQVLAPPGVVASIAFPEARPELRLLQFLAAATETPVTPFHLQLDWASPLETILDVLVSLVRAHAWEDIALVLCRVRDPGSLVTLWTNHASQAPKFVLDLSRLDSRNDSLRAGLALLGALEGGGTPVPAAVLLGCSTARAHEVLEAAPPGPQWLLGTPLPAEALPTTGLPPGVLALGETEQHSLEAVVHDMVELVAQALSSMALVHPERALLPAVVNCDDLKTGGSEATGRTLARFLGNTSFQGRTGAVWVTGSSQVHVSRHFKVWSLRRDPLGAPAWATVGSWQDGQLDFQPGAAALRVPSPSGTQARPKLRVVTLVEHPFVFTRESDEDGQCPAGQLCLDPGTNDSARLDALFAALVNGSVPRTLRRCCYGYCIDLLERLAEDLAFDFELYIVGDGKYGALRDGRWTGLVGDLLAGRAHMAVTSFSINSARSQVVDFTSPFFSTSLGIMVRTRDTASPIGAFMWPLHWSMWVGVFAALHLTALFLTLYEWRSPYGLTPRGRNRGTVFSYSSALNLCYAILFGRTVSSKTPKCPTGRFLMNLWAIFCLLVLSSYTANLAAVMVGDKTFEELSGIHDPKLHHPSQGFRFGTVWESSAEAYIKASFPEMHAHMRRHSAPTTPHGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMGVYHFSGLFVLLCLGLGSALLTSLGEHVFYRLVLPRIRRGNKLQYWLHTSQKIHRALNTGPPEGQQERAEQERSGPKDELPATDGAGRWRRVRRAVERERRVRFLLEPGEAGGDRPWLCSNGPGLQAELRELELRIEAAREQLRSALLRRGELRALLGDGTRLRPLRLLHAAPAES

SEQ ID NO: 3 - DNA, 3178 bp, Rattus sp.
  CDS (76)...(3081)
gcacgaggggacaagagcgggtctggctggggtgtctccttcccttcgcacagcacagtggtaacttctttcgggatggagagtgtgcggacgctgtggctcagcgtggccctggcgctggcggtggggtcccgagtggtgcgcggtcaccctcagccctgccgggttcccacgcgcgctggggcctccgtgcgcctggcggcgctcctgccccgggcgcccgccgcccgcgcccgcgtcctagctgccctggccacccctgcgccgcggctgccgcacaacctgagtctggaactggtggccgtcgcgtccccgacccgggaccccgcgtcgctagctcgaggtctgtgccaggttctggcaccgcctggcgtggtggcctctatagcctttcccgaggcgcggcccgagctgcggctactgcagttcctggcagccgccacagagaccccagtggtgagcgtcctgcggagggaggtgcgcacggccctcggagccccgactccgttccatctgcagctggactgggctagtcccctggagaccatactggatgtgctggtgtccctggtacgggcacatgcctgggaggacattgctctagtactctgccgtgtccgggaccctggcagcctggtgacactctggactaaccatgctagccaggctccaaagtttgtgctggacctgagccggctggacagcaggaatgacagccttcgggctggactggccctgttgggggcgctggaaggagggggaaccccagtgcctgcagcagtcctcctaggctgcagcactgcccgtgcacatgaggtcctagaggctgcaccaccgggtccccagtggttgctgggcacaccattgcccgctgaggcactgcccacgactggtctgccacctggcgtgctggcgctgggggaaaccgaacaacactctctggaagctgtcgtccacgacatggtggagcttgtggctcaggcactcagtagcatggcccttgtacacccagagcgggcactgcttccagctgtggtgaactgtgatgacctgaaaacaggcggatctgaggcaacagggcgcaccttggctcggtttctcggcaacacctcatttcagggccgaacaggggccgtgtgggtgacaggctcctctcaggtgcatgtgtctcggcatttcaaggtatggagcctgcgccgggatccgctgggtgccccagcctgggcaaccgtgggcagctggcaggatggacagctggacttccagccaggggcagccgctctccgagtcccatctccgtctggcacccaggcccgaccaaagctgcgtgtggtaaccctggtggaacacccgtttgtgttcaccagggaatctgatgaagacggacagtgcccagctgggcagctgtgtctggacccaggcaccaatgactcagccaggctggatgccctctttgctgcactggtgaatggctcagtacctcgaacgctgagaagatgctgctatggctactgcatcgacctgctggagcggctggccgaggacctggcctttgactttgagctctatattgtgggggatggcaagtacggggccctgcgtgatgggcgctggacgggcctggtgggtgacctgctggctggccgggcacacatggctgtgaccagcttcagcatcaactcagctcgctctcaggtggtggatttcaccagccctttcttctccaccagcctggggattatggtgcgcacgagagacacggcctcgcccatcggggctttcatgtggcccctgcactggtccatgtgggtgggcgtgtttgctgctctgcacctcacagcgctctttctcaccctgtacgaatggcgaagtccctacgggctcacgccgcgcggccgcaaccgtggcactgtcttctcttactcctccgcgctcaacctgtgctatgccattctctttggacgcactgtctccagtaagacgcccaagtgccctactggacgcttcctcatgaacctctgggcaatcttctgcctgctggtgctttccagttacacggccaacctggctgctgtcatggttggggacaaaacctttgaggagctgtctggaatccatgatcccaagctgcaccacccttcccaaggctttcgctttggcaccgtatgggagagcagcgcggaggcctacatcaaggcaagcttccctgagatgcacgcacacatgcgtcggcacagcgcacccaccactccacatggggtggccatgctcacgagcgacccgcccaagctcaacgccttcatcatggataaatcactactggactatgaggtgtccatagatgcggactgcaagctgctcaccgttggcaaaccctttgctatcgagggctacggcatagggctaccccaaaactcgccgctcacctccaacctgtcggagttcatcagtaggtacaagtcttcaggcttcattgatctgctccatgacaagtggtacaagatggtgccttgcgggaagcgggtgttcgccgtgacggagacgctgcagatgggggtctaccacttctcaggattgtttgtcctgctgtgcctcgggctgggcagcgcgcttctcacctctctgggtgagcatgtcttctaccgcctggtgctgccgcgcatccgcaggggtaataagctgcagtattggcttcacacgagccagaagatccaccgagccctcaatacaggaccacccgaggggcaacaggagagggcagagcaggagcgcagcggccccaaggacgagctgcctgccaccgatggtgcagggcgctggaggcgggtgcgccgggctgtggaacgggagcgacgcgtgcgtttcctgctggaacctggggaggctggcggagaccgcccgtggctctgctccaacgggcccgggctgcaagcggagctgcgggagctggagctgcgcattgaggctgcacgggagcagctgcgcagtgcgctgttgcggcgcggggagctgcgggccctgcttggggatggcacccggctcaggccactgcgcctgttgcatgcggcgcctgctgagagctgaggaaccacaaggccgcactgtccacgacagtttattctatatacaaacacgactctgtacactgcaattaaatagcgtggaacgtgaaaaaaaa

SEQ ID NO: 4 - protein, 1002 aa, Rattus sp.
MESVRTLWLSVALALAVGSRVVRGHPQPCRVPTRAGASVRLAALLPRAPAARARVLAALATPAPRLPHNLSLELVAVASPTRDPASLARGLCQVLAPPGVVASIAFPEARPELRLLQFLAAATETPVVSVLRREVRTALGAPTPFHLQLDWASPLETILDVLVSLVRAHAWEDIALVLCRVRDPGSLVTLWTNHASQAPKFVLDLSRLDSRNDSLRAGLALLGALEGGGTPVPAAVLLGCSTARAHEVLEAAPPGPQWLLGTPLPAEALPTTGLPPGVLALGETEQHSLEAVVHDMVELVAQALSSMALVHPERALLPAVVNCDDLKTGGSEATGRTLARFLGNTSFQGRTGAVWVTGSSQVHVSRHFKVWSLRRDPLGAPAWATVGSWQDGQLDFQPGAAALRVPSPSGTQARPKLRVVTLVEHPFVFTRESDEDGQCPAGQLCLDPGTNDSARLDALFAALVNGSVPRTLRRCCYGYCIDLLERLAEDLAFDFELYIVGDGKYGALRDGRWTGLVGDLLAGRAHMAVTSFSINSARSQVVDFTSPFFSTSLGIMVRTRDTASPIGAFMWPLHWSMWVGVFAALHLTALFLTLYEWRSPYGLTPRGRNRGTVFSYSSALNLCYAILFGRTVSSKTPKCPTGRFLMNLWAIFCLLVLSSYTANLAAVMVGDKTFEELSGIHDPKLHHPSQGFRFGTVWESSAEAYIKASFPEMHAHMRRHSAPTTPHGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMGVYHFSGLFVLLCLGLGSALLTSLGEHVFYRLVLPRIRRGNKLQYWLHTSQKIHRALNTGPPEGQQERAEQERSGPKDELPATDGAGRWRRVRRAVERERRVRFLLEPGEAGGDRPWLCSNGPGLQAELRELELRIEAAREQLRSALLRRGELRALLGDGTRLRPLRLLHAAPAES

SEQ ID NO: 5 - DNA, 3096 bp, Homo sapiens
  CDS (1)...(3033)
  misc_feature 2692, 2693, 2694, 2695, 2696, 2697, 2698, 2699, 2700,2701, 2702, 2703, 2704, 2705, 2706, 2707, 2708, 2709, 2710, 2711,2712, 2713, 2714, 2715, 2716, 2717, 2718, 2719, 2720, 2721,2722, 2723, 2724, 2725, 2726, 2727, 2728, 2729, 2730: n = A,T,C or G
  misc_feature 2791, 2792, 2793, 2794, 2795, 2796, 2797, 2798, 2799,2800, 2801, 2802, 2803, 2804, 2805, 2806, 2807, 2808, 2809, 2810,2811, 2812, 2813, 2814, 2827, 2828, 2829, 2830, 2831, 2832,2833, 2834, 2835, 2836, 2837, 2838, 2839, 2840, 2841: n = A,T,C or G
  misc_feature 2842, 2843, 2844, 2845, 2846, 2847, 3037, 3038, 3039: n = A,T,C or G
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagctgcaccacccggcgcagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccaggnnnnnnnnnnnnnnnnnnnnnnnnnnnnnnnnnnnnnnntggaaacgggcgcgccgggccgtggacaaggagcgccgcgtgcgcttcctgctggagcccnnnnnnnnnnnnnnnnnnnnnnnntggctgtgctccnnnnnnnnnnnnnnnnnnnnngagctgcaggagctggagcgccgcatcgaagtcgcgcgtgagcggctccgccaggccctggtgcggcgcggccagctcctggcacagctcggggacagcgcacgtcaccggcctcggcgcttgcttcaggccagagcggcccccgcggaggccccaccacactctggccgaccggggagccaggaatgannngacagtttattctatatacaaacacaattttgtacactgcaattaaatagaatggaa

SEQ ID NO: 6 - protein, 1011 aa, Homo sapiens
  VARIANT 898, 899, 900, 901, 902, 903, 904, 905, 906, 907, 908,909, 910, 931, 932, 933, 934, 935, 936, 937, 938, 943, 944, 945,946, 947, 948, 949: Xaa = Any Amino Acid
MEFVRALWLGLALALGPGSAGGHPQPCGVLARLGGSVRLGALLPRAPLARARARAALARAALAPRLPHNLSLELVVAAPPARDPASLTRGLCQALVPPGVAALLAFPEARPELLQLHFLAAATETPVLSLLRREARAPLGAPNPFHLQLHWASPLETLLDVLVAVLQAHAWEDVGLALCRTQDPGGLVALWTSRAGRPPQLVLDLSRRDTGDAGLRARLAPMAAPVGGEAPVPAAVLLGCDIARARRVLEAVPPGPHWLLGTPLPPKALPTAGLPPGLLALGEVARPPLEAAIHDIVQLVARALGSAAQVQPKRALLPAPVNCGDLQPAGPESPGRFLANTSFQGRTGPVWVTGSSQVHMSRHFKVWSLRRDPRGAPAWATVGSWRDGQLDLEPGGASARPPPPQGAQVWPKLRVVTLLEHPFVFARDPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKLHHPAQGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEPRXXXXXXXXXXXXXWKRARRAVDKERRVRFLLEPXXXXXXXXWLCSXXXXXXXELQELERRIEVARERLRQALVRRGQLLAQLGDSARHRPRRLLQARAAPAEAPPHSGRPGSQE

SEQ ID NO: 7 - DNA, 3166 bp, Mus musculus
  CDS (63)...(3077)
agagcggggctggctgggatgtctccttaccctcgcacagcacagtggtaacttccatcgggatggagtgtgtgcagacgctgtggctcagcctggccctggcgctggcgcgagggtcctgggtggtgcgcggtcaccctcagccctgcggggttcccacgcgcgccggggcctccgtgcgcctggctgcgctcctaccccgggcgcccgccgcccgcgcccgcgtcctagccgccctggccaccccttccccgcggctgccgcacaacctgagtctggagctagtggccgtcgcgtccccaacccgggaccccgcgtcgctggcccgaggtctgtgccaggttctggcaccgcccggcgtggtggcctctataacctttcccgaggcgcggcctgagctacggctattgcagttcctggcagctgccacagagaccccggtgctgagcgtcctacggagggaggtgcgcgcgcccctcggagctcggcggaccccgttccacctgcagctggactgggctagtcccctggagaccatcctggatgtgctggtgtccctggtacgggcacacgcctgggaggacattgctctagtgctctgtcgtgtccgggaccccagtggcctggtgacactctggaccagccgtgctagccaggctccaaagtttgtgctggacctgagccagttggacagcgggaatgacagccttcgggctacactggccctgctggggacgctggaaggagggggaacccccgtgtctgcagccgtcctcctgggctgcagcactgcccatgcacatgaggtcctagaggcagcaccaccgggtccccagtggctgctgggcacaccactgcccgccgaggcactgcccaaaaccggtctgccccctggggtgttggtgctgggggaaaccgggcagccttccctggaagctgccgtccacgacatggtggagcttgtggctcgggcactcagcagcatggccctcatgcacccagagcgggccctgcttccagcggcagtaaattgtgaggacctgaaaacgggcggctctgagtcaacagcacgcaccttggctaggtggtttctgagcaacacctcatttcagggccgcacaggggctgtgtgggtggcaggctcctctcaggtgcatgtgtctcggcatttcaaggtatggagcttacgcagggacccgctgggtgccccagcctgggcaacagtaggcagctggcaggatggacagctggacttccagccaggggcggctgctctccgagttccatctccatctggcacccaggcccggccaaagctgcgagtggtaactctggtggaacacccatttgtgttcaccagggaatctgatgaagatgggcagtgcccggctgggcagctgtgtctagacccaggcaccaatgactcggccaggctggatgcgctcttcactgcattggagaatggctccgtgcctcgcaccctgagaagatgctgttatggctactgcattgacctgctggagcggctggccgaggacctggcctttgactttgagctctatattgtgggggatggcaagtacggggccctgcgtgatggacgctggacaggcctggtgggtgacctgctggccggtcgggcacacatggccgtgaccagcttcagtatcaactcggctcgctcccaggtggtggatttcaccagccctttcttctccaccagcctgggcatcatggtgcgcacgcgagatacagcctcacccattggggctttcatgtggcccctgcactggtccatgtgggtgggtgtgtttgctgccctgcacctcacagcgctcttccttactctgtacgaatggcggagtccctacgggctcacgccacgcggccgcaaccgtggtaccgtcttctcctactcctccgctctcaacctctgctacgccattctcttcggacgcactgtctccagtaagacacccaagtgtcctaccggacgcttcctcatgaatctctgggcaatcttctgcctgctggtgctatccagttacacagccaacctggcagctgtcatggtcggggacaagacatttgaggagctgtctggaatccatgatcccaagctgcaccacccttcccaaggcttccgctttggcaccgtgtgggagagcagcgcggaggcctacatcaaggcgagcttccccgagatgcacgcacacatgcgtcgccacagcgcacctaccactccacacggagtggccatgctcacgagcgacccgcccaagctcaacgccttcatcatggataaatcactactggattatgaggtgtccatagatgcggactgcaagctgctcaccgtgggcaaaccctttgcgatcgagggctacggcatagggctgccccaaaattcgccgctcacctccaacctgtcagagttcatcagtaggtacaagtcctcaggcttcattgatctgctccatgacaagtggtacaagatggtgccttgcgggaagcgggtgttcgccgtgacggagacgctgcagatgggggtctaccacttgtcagggttgtttgtcctgctgtgcctcgggctgggcagtgcacttctcacctcgctgggtgagcacgtcttctaccgcctggtgctgccgcgcatccgcaggggcaataagctgcagtattggcttcacacgagccaggaagatccaccgagccctcaacacagggccaccagaggggcaacaggagagggcagagcaggagtgcagggccccaaggaggagcaacctgcagccgacggtgcggggcgctggaggcgggtgcgccgggccgtggtggaacgggaacggcgcgtgcgtttcctgctggaacctggggaggctggcggggaccatccgtggctctgctccaatgggcccggggtgcaagcagaactgcgggagctggagctgcgcattgaggctgcacgggagcggctgcgtagtgcgctgctgcggcgaggggaactgcgggcccagcttggggatggcacccggctcaggccactgcgcctgctgcatgcggcgcccgccgagagctgaggaactacacggccacactgtccacgacagtttattctatatacaaacacgactctgtacactgcaattaaatagcgtggaacgtg

SEQ ID NO: 8 - protein, 1005 aa, Mus musculus
MECVQTLWLSLALALARGSWVVRGHPQPCGVPTRAGASVRLAALLPRAPAARARVLAALATPSPRLPHNLSLELVAVASPTRDPASLARGLCQVLAPPGVVASITFPEARPELRLLQFLAAATETPVLSVLRREVRAPLGARRTPFHLQLDWASPLETILDVLVSLVRAHAWEDIALVLCRVRDPSGLVTLWTSRASQAPKFVLDLSQLDSGNDSLRATLALLGTLEGGGTPVSAAVLLGCSTAHAHEVLEAAPPGPQWLLGTPLPAEALPKTGLPPGVLVLGETGQPSLEAAVHDMVELVARALSSMALMHPERALLPAAVNCEDLKTGGSESTARTLARWFLSNTSFQGRTGAVWVAGSSQVHVSRHFKVWSLRRDPLGAPAWATVGSWQDGQLDFQPGAAALRVPSPSGTQARPKLRVVTLVEHPFVFTRESDEDGQCPAGQLCLDPGTNDSARLDALFTALENGSVPRTLRRCCYGYCIDLLERLAEDLAFDFELYIVGDGKYGALRDGRWTGLVGDLLAGRAHMAVTSFSINSARSQVVDFTSPFFSTSLGIMVRTRDTASPIGAFMWPLHWSMWVGVFAALHLTALFLTLYEWRSPYGLTPRGRNRGTVFSYSSALNLCYAILFGRTVSSKTPKCPTGRFLMNLWAIFCLLVLSSYTANLAAVMVGDKTFEELSGIHDPKLHHPSQGFRFGTVWESSAEAYIKASFPEMHAHMRRHSAPTTPHGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMGVYHLSGLFVLLCLGLGSALLTSLGEHVFYRLVLPRIRRGNKLQYWLHTSQEDPPSPQHRATRGATGEGRAGVQGPKEEQPAADGAGRWRRVRRAVVERERRVRFLLEPGEAGGDHPWLCSNGPGVQAELRELELRIEAARERLRSALLRRGELRAQLGDGTRLRPLRLLHAAPAES

SEQ ID NO: 9 - DNA, 2691 bp, Homo sapiens
  CDS (1)...(2691)
atggagtttgtgcgggcgctgtggctgggcctggcgctggcgctggggccggggtccgcggggggccaccctcagccgtgcggcgtcctggcgcgcctcgggggctccgtgcgcctgggcgccctcctgccccgcgcgcctctcgcccgcgcccgcgcccgcgccgccctggcccgggccgccctggcgccgcggctgccgcacaacctgagcttggagctggtggtcgccgcgccccccgcccgcgaccccgcctcgctgacccgcggcctgtgccaggcgctggtgcctccgggcgtggcggccctgctcgcctttcccgaggctcggcccgagctgctgcagctgcacttcctggcggcggccaccgagacccccgtgctcagcctgctgcggcgggaggcgcgcgcgcccctcggagccccgaacccattccacctgcagctgcactgggccagccccctggagacgctgctggatgtgctggtggcggtgctgcaggcgcacgcctgggaagacgtcggcctggccctgtgccgcactcaggaccccggcggcctggtggccctctggacaagccgggctggccggcccccacagctggtcctggacctaagccggcgggacacgggagatgcaggactgcgggcacgcctggccccgatggcggcgccagtggggggtgaagcaccggtacccgcggcggtcctcctcggctgtgacatcgcccgtgcccgtcgggtgctggaggccgtacctcccggcccccactggctgttggggacaccactgccgcccaaggccctgcccaccgcggggctgccaccagggctgctggcgctgggcgaggtggcacgacccccgctggaggccgccatccatgacattgtgcaactggtggcccgggcgctgggcagtgcggcccaggtgcagccgaagcgagccctcctccccgccccggtcaactgcggggacctgcagccggccgggcccgagtccccggggcgcttcttggccaacacgtccttccagggccgcacgggccccgtgtgggtgacaggcagctcccaggtacacatgtctcggcactttaaggtgtggagccttcgccgggacccacggggcgccccggcctgggccacggtgggcagctggcgggacggccagctggacttggaaccgggaggtgcctctgcacggcccccgcccccacagggtgcccaggtctggcccaagctgcgtgtggtaacgctgttggaacacccatttgtgtttgcccgtgatccagacgaagacgggcagtgcccagcggggcagctgtgcctggaccctggcaccaacgactcggccaccctggacgcactgttcgccgcgctggccaacggctcagcgccccgtgccctgcgcaagtgctgctacggctactgcattgacctgctggagcggctggcggaggacacgcccttcgacttcgagctgtacctcgtgggtgacggcaagtacggcgccctgcgggacggccgctggaccggcctggtcggggacctgctggccggccgggcccacatggcggtcaccagcttcagtatcaactccgcccgctcacaggtggtggacttcaccagccccttcttctccaccagcctgggcatcatggtgcgggcacgggacacggcctcacccatcggtgcctttatgtggcccctgcactggtccacgtggctgggcgtctttgcggccctgcacctcaccgcgctcttcctcaccgtgtacgagtggcgtagcccctacggcctcacgccacgtggccgcaaccgcagcaccgtcttctcctactcctcagccctcaacctgtgctacgccatcctcttcagacgcaccgtgtccagcaagacgcccaagtgccccacgggccgcctgctcatgaacctctgggccatcttctgcctgctggtgctgtccagctacacggccaacctggctgccgtcatggtcggggacaagaccttcgaggagctgtcggggatccacgaccccaagctgcaccacccggcgcagggcttccgcttcggcaccgtgtgggagagcagcgccgaggcgtacatcaagaagagcttccccgacatgcacgcacacatgcggcgccacagcgcgcccaccacgccccgcggcgtcgccatgctcacgagcgacccccccaagctcaacgccttcatcatggacaagtcgctcctggactacgaggtctccatcgacgccgactgcaaactgctgaccgtgggaaagcccttcgccattgagggctatgggatcggactgccccagaactcgccgctcacctccaacctgtccgagttcatcagccgctacaagtcctccggcttcatcgacctgctccacgacaagtggtacaagatggtgccttgcggcaagcgggtctttgcggttacagagaccctgcagatgagcatctaccacttcgcgggcctcttcgtgttgctgtgcctgggcctgggcagcgctctgctcagctcgctgggcgagcacgccttcttccgcctggcgctgccgcgcatccgcaaggggagcaggctgcagtactggctgcacaccagccagaaaatccaccgcgccctcaacacggagccaccagaggggtcgaaggaggagacggcagaggcggagcccagg

SEQ ID NO: 10 - protein, 897 aa, Homo sapiens
MEFVRALWLGLALALGPGSAGGHPQPCGVLARLGGSVRLGALLPRAPLARARARAALARAALAPRLPHNLSLELVVAAPPARDPASLTRGLCQALVPPGVAALLAFPEARPELLQLHFLAAATETPVLSLLRREARAPLGAPNPFHLQLHWASPLETLLDVLVAVLQAHAWEDVGLALCRTQDPGGLVALWTSRAGRPPQLVLDLSRRDTGDAGLRARLAPMAAPVGGEAPVPAAVLLGCDIARARRVLEAVPPGPHWLLGTPLPPKALPTAGLPPGLLALGEVARPPLEAAIHDIVQLVARALGSAAQVQPKRALLPAPVNCGDLQPAGPESPGRFLANTSFQGRTGPVWVTGSSQVHMSRHFKVWSLRRDPRGAPAWATVGSWRDGQLDLEPGGASARPPPPQGAQVWPKLRVVTLLEHPFVFARDPDEDGQCPAGQLCLDPGTNDSATLDALFAALANGSAPRALRKCCYGYCIDLLERLAEDTPFDFELYLVGDGKYGALRDGRWTGLVGDLLAGRAHMAVTSFSINSARSQVVDFTSPFFSTSLGIMVRARDTASPIGAFMWPLHWSTWLGVFAALHLTALFLTVYEWRSPYGLTPRGRNRSTVFSYSSALNLCYAILFRRTVSSKTPKCPTGRLLMNLWAIFCLLVLSSYTANLAAVMVGDKTFEELSGIHDPKLHHPAQGFRFGTVWESSAEAYIKKSFPDMHAHMRRHSAPTTPRGVAMLTSDPPKLNAFIMDKSLLDYEVSIDADCKLLTVGKPFAIEGYGIGLPQNSPLTSNLSEFISRYKSSGFIDLLHDKWYKMVPCGKRVFAVTETLQMSIYHFAGLFVLLCLGLGSALLSSLGEHAFFRLALPRIRKGSRLQYWLHTSQKIHRALNTEPPEGSKEETAEAEPR

SEQ ID NO: 11 - DNA, 1786 bp, Mus musculus
  CDS (2)...(1444)
catcaactgcttagagcatgttcgagatcgatggccgcgggagggtgtcctgcgggtggaggtgcgccacaactcgagccgggcaccggtgatcctgcagttctgtgatgggggcctcggtggcctggagctggaacccgggggcctggagctggaggaggaggagctcacagtggagatgttcaccaacagctccatcaagtttgagctggacattgagcccaaggtgttcaagccacagagcggtgcagatgccctgaacgacagccaggacttcccttttcctgagacgccagcaaaagtgtggccacaggatgagtacattgtggagtactcactggaatatggcttcctgcggctatcccaagccacacgccagcgtctgagcattcctgtcatggtggtcaccctagaccccacgcgggaccagtgctttggggaccgcttcagccgcctattgctggatgagttcctgggctatgatgacatcctcatgtccagtgtaaagggtctggcagagaacgaggagaacaaaggcttcttgaggaatgtggtctctggggagcactaccgctttgtcagcatgtggatggcgcgcacatcctacctggcggcctttgtcatcatggtcatctttaccctcagcgtgtccatgctgttgcgatactcgcaccaccagatcttcgtcttcatcgtggacctgctgcagatgctggagatgaacatggccatcgccttccccgcagcgcccttgctgaccgtcatcctggctctcgtcgggatggaagccatcatgtctgagttcttcaatgataccaccacggccttctacatcatcctcactgtgtggctggccgaccagtatgatgccatctgctgccacaccaacaccagcaagcggcactggctgaggttcttctacctctaccacttcgccttctatgcctaccactaccgctttaacgggcagtacagcagcctggccctggtcacctcctggctcttcatccagcattccatgatctacttcttccaccactatgagttgcccgccatcctgcagcagatccgaatccaggagatgctgcttcagacgccaccgctgggccccgggacccccacggcgctgcctgacgacctcaacaacaactctggctcccctgccactccggatcccagccctcccctcgcgctgggccccagctccagccccgcgcccactggcggggcatctgggcctggctcactgggcgctggggcctcagtatccggcagtgacctaggttgggtggccgagaccgccgccatcatctctgacgcatccttcctgtcggggctgagcgcctctctcctggagcggcggccaacagcccctagtaccccggacagctcacgacctgaccctggggtccctctggaggacgcacccgcccctgccgggtcctgagaccggtgtcgtgcccggctcccagtggagccgcggcccgagcccaagagagctgtggtctgcagggagaggggctggtggcgaaggttctggaagcggccgggacagggcggcgatgggcacgaggccatcggccgcctggtgctccccagtgccttccacacggcgcccgcacggggccgagcgcccgggcccggactagcaggtggtggctcacgatgcggggcacacgttccacgctatttaattgcagtgtacagagtcgtgtttgtatatagaataaactgtcgtggacagtgaaaaaaaaaaaaaaaaaaaaaaaaaaaaaaaaaaaaaaaa

SEQ ID NO: 12 - protein, 481 aa, Mus musculus
INCLEHVRDRWPREGVLRVEVRHNSSRAPVILQFCDGGLGGLELEPGGLELEEEELTVEMFTNSSIKFELDIEPKVFKPQSGADALNDSQDFPFPETPAKVWPQDEYIVEYSLEYGFLRLSQATRQRLSIPVMVVTLDPTRDQCFGDRFSRLLLDEFLGYDDILMSSVKGLAENEENKGFLRNVVSGEHYRFVSMWMARTSYLAAFVIMVIFTLSVSMLLRYSHHQIFVFIVDLLQMLEMNMAIAFPAAPLLTVILALVGMEAIMSEFFNDTTTAFYIILTVWLADQYDAICCHTNTSKRHWLRFFYLYHFAFYAYHYRFNGQYSSLALVTSWLFIQHSMIYFFHHYELPAILQQIRIQEMLLQTPPLGPGTPTALPDDLNNNSGSPATPDPSPPLALGPSSSPAPTGGASGPGSLGAGASVSGSDLGWVAETAAIISDASFLSGLSASLLERRPTAPSTPDSSRPDPGVPLEDAPAPAGS

SEQ ID NO: 13 - DNA, 579 bp, Homo sapiens
ccacgcgtccgatcttgtaccacttgtcgtggagcaggtcgatgaagccggaggacttgtagcggctgatgaactcggacaggttggaggaggtccccagagaccccggcgccccgcctcgcccaggtgcctctcaccctcaatggcgaagggctttcccacggtcagcagtttgcagtcggcgtcgatggagacctcgtagtccaggagcgacttgtccatgatgaaggcgttgagcttgggggggtcgctcctgctggggccgggggcgggggtcagccatcgccccgcccaccccacgccccgcccccgcctcaccccgcgcccgggctcacgtgagcatggcgacyccgcggggcgtggtgggcgcgctgtggcgccgcatgtgtgcgtgcatgtcggggaagctcttcttgatgtacgcctcggcgctgttctcccacacggtgccgaagcggaagccctgcgccgggtggtgcagctgcgcgggggaccccgtcagcgcctctgctgcccctcaggacccctgaccattgaggggcgcgccgttctccggggtggggccgtcctgggcacttg

SEQ ID NO: 14 - protein, 938 aa, Rattus sp.
MSTMHLLTFALLFSCSFARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYNWNHIILLVSDDHEGRAAQKRLETLLEERESKAEKVLQFDPGTKNVTALLMEARELEARVIILSASEDDAATVYRAAAMLNMTGSGYVWLVGEREISGNALRYAPDGIIGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTMSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQDRKSGRAEPDPKKKATFRAITSTLASSFKRRRSSKDTSTGGGRGALQNQKDTVLPRRAIEREEGQLQLCSRHRES

SEQ ID NO: 15 - protein, 1464 aa, Rattus sp.
MGRLGYWTLLVLPALLVWRDPAQNAAAEKGPPALNIAVLLGHSHDVTERELRNLWGPEQATGLPLDVNVVALLMNRTDPKSLITHVCDLMSGARIHGLVFGDDTDQEAVAQMLDFISSQTFIPILGIHGGASMIMADKDPTSTFFQFGASIQQQATVMLKIMQDYDWHVFSLVTTIFPGYRDFISFIKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGLGILTTAASSMLEKFSYIPEAKASCYGQAEKPETPLHTLHQFMVNVTWDGKDLSFTEEGYQVHPRLVVIVLNKDREWEKVGKWENQTLSLRHAVWPRYKSFSDCEPDDNHLSIVTLEEAPFVIVEDIDPLTETCVRNTVPCRKFVKINNSTNEGMNVKKCCKGFCIDILKKLSRTVKFTYDLYLVTNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTRFNQRGVEDALVSLKTGKLDAFIYDAAVLNYKAGRDEGCKLVTIGSGYIFATTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISNMSNMNSSRMDSPKRATDFIQRGSLIVDMVSDKGNLIYSDNRSFQGKDSIFGDNMNELQTFVANRHKDNLSNYVFQGQHPLTLNDSNPNTVEVAVSTESKGNSRPRQLWKKSMESLRQDSLNQNPVSQRDEKTAENRTHSLKSPRYLPEEVAHSDISETSSRATCHREPDNNKNHKTKDNFKRSMASKYPKDCSDVDRTYMKTKASSPRDKIYTIDGEKEPSFHLDPPQFVENITLPENVGFPDTYQDHNENFRKGDSTLPMNRNPLHNEDGLPNNDQYKLYAKHFTLKDKGSPHSEGSDRYRQNSTHCRSCLSNLPTYSGHFTMRSPFKCDACLRMGNLYDIDEDQMLQETGNPATREEVYQQDWSQNNALQFQKNKLRINRQHSYDNILDKPREIDLSRPSRSISLKDRERLLEGNLYGSLFSVPSSKLLGNKSSLFPQGLEDSKRSKSLLPDHASDNPFLHTYGDDQRLVIGRCPSDPYKHSLPSQAVNDSYLRSSLRSTASYCSRDSRGHSDVYISEHVMPYAANKNTMYSTPRVLNSCSNRRVYKKMPSIESDV

SEQ ID NO: 16 - protein, 1115 aa, Rattus sp.
MRRLSLWWLLSRVCLLLPPPCALVLAGVPSSSSHPQPCQILKRIGHAVRVGAVHLQPWTTAPRAASRAQEGGRAGAQRDDPESGTWRPPAPSQGARWLGSALHGRGPPGSRKLGEGAGAETLWPRDALLFAVENLNRVEGLLPYNLSLEVVMAIEAGLGDLPLMPFSSPSSPWSSDPFSFLQSVCHTVVVQGVSALLAFPQSQGEMMELDLVSSVLHIPVLSIVRHEFPRESQNPLHLQLSLENSLSSDADVTVSILTMNNWYNFSLLLCQEDWNITDFLLLTENNSKFHLESVINITANLSSTKDLLSFLQVQMDNIRNSTPTMVMFGCDMDSIRQIFEMSTQFGLSPPELHWVLGDSQNVEELRTEGLPLGLIAHGKTTQSVFEYYVQDAMELVARAVATATMIQPELALLPSTMNCMDVKTTNLTSGQYLSRFLANTTFRGLSGSIKVKGSTIISSENNFFIWNLQHDPMGKPMWTRLGSWQGGRIVMDSGIWPEQAQRHKTHFQHPNKLHLRVVTLIEHPFVFTREVDDEGLCPAGQLCLDPMTNDSSMLDRLFSSLHSSNDTVPIKFKKCCYGYCIDLLEQLAEDMNFDFDLYIVGDGKYGAWKNGHWTGLVGDLLSGTANMAVTSFSINTARSQVIDFTSPFFSTSLGILVRTRDTAAPIGAFMWPLHWTMWLGIFVALHITAIFLTLYEWKSPFGMTPKGRNRNKVFSFSSALNVCYALLFGRTAAIKPPKCWTGRFLMNLWAIFCMFCLSTYTANLAAVMVGEKIYEELSGIHDPKLHHPSQGFRFGTVRESSAEDYVRQSFPEMHEYMRRYNVPATPDGVQYLKNDPEKLDAFIMDKALLDYEVSIDADCKLLTVGKPFAIEGYGIGLPPNSPLTSNISELISQYKSHGFMDVLHDKWYKVVPCGKRSFAVTETLQMGIKHFSGLFVLLCIGFGLSILTTIGEHIVHRLLLPRIKNKSKLQYWLHTSQRFHRALNTSFVEEKQPRSKTKRVEKRSNLGPQQLMVWNTSNLSHDNQRKYIFNDEEGQNQLGTQAHQDIPLPQRRRELPASLTTNGKADSLNVTRSSVIQELSELEKQIQVIRQELQLAVSRKTELEEYQKTNRTCES

SEQ ID NO: 17 - DNA, 21 bp, Artificial Sequence
  synthetic oligonucleotide
tgctgctatggctactgcatc

SEQ ID NO: 18 - DNA, 23 bp, Artificial Sequence
  synthetic oligonucleotide
atgacagcagccaggttggccgt

SEQ ID NO: 19 - DNA, 20 bp, Artificial Sequence
  synthetic oligonucleotide
cacacatggctgtgaccagc

SEQ ID NO: 20 - DNA, 21 bp, Artificial Sequence
  synthetic oligonucleotide
agaatggcatagcacaggttg

SEQ ID NO: 21 - protein, 6 aa, Rattus sp.
ADGKFG

SEQ ID NO: 22 - protein, 6 aa, Rattus sp.
TNGKHG

SEQ ID NO: 23 - protein, 6 aa, Rattus sp.
TNGKHG

SEQ ID NO: 24 - protein, 6 aa, Rattus sp.
TNGKHG

SEQ ID NO: 25 - protein, 6 aa, Rattus sp.
TNGKHG

SEQ ID NO: 26 - protein, 6 aa, Rattus sp.
GDGKYG

SEQ ID NO: 27 - protein, 6 aa, Rattus sp.
GDGKYG

SEQ ID NO: 28 - protein, 6 aa, Rattus sp.
GDGKYG

SEQ ID NO: 29 - protein, 11 aa, Rattus sp.
APLTINNERAQ

SEQ ID NO: 30 - protein, 11 aa, Rattus sp.
GSLTINEERSE

SEQ ID NO: 31 - protein, 11 aa, Rattus sp.
GSLTINEERSE

SEQ ID NO: 32 - protein, 11 aa, Rattus sp.
GSLTINEERSE

SEQ ID NO: 33 - protein, 11 aa, Rattus sp.
GSLTINEERSE

SEQ ID NO: 34 - protein, 11 aa, Rattus sp.
TSFSINTARSQ

SEQ ID NO: 35 - protein, 11 aa, Rattus sp.
TSFSINSARSQ

SEQ ID NO: 36 - protein, 11 aa, Rattus sp.
APLTITLVREE

SEQ ID NO: 37 - protein, 9 aa, Rattus sp.
ATVKQSSVD

SEQ ID NO: 38 - protein, 9 aa, Rattus sp.
GTVPNGSTE

SEQ ID NO: 39 - protein, 9 aa, Rattus sp.
GTVPNGSTE

SEQ ID NO: 40 - protein, 9 aa, Rattus sp.
GTVPNGSTE

SEQ ID NO: 41 - protein, 9 aa, Rattus sp.
GTVPNGSTE

SEQ ID NO: 42 - protein, 9 aa, Rattus sp.
GTVRESSAE

SEQ ID NO: 43 - protein, 9 aa, Rattus sp.
GTVWESSAE

SEQ ID NO: 44 - protein, 9 aa, Rattus sp.
GTLDSGSTK

SEQ ID NO: 45 - protein, 25 aa, Rattus sp.
DALTLSSAMWFSWGVLLNSGIGEGA

SEQ ID NO: 46 - protein, 25 aa, Rattus sp.
PSFTIGKAIWLLWGLVFNNSVPVQN

SEQ ID NO: 47 - protein, 25 aa, Rattus sp.
PSFTIGKAIWLLWGLVFNNSVPVQN

SEQ ID NO: 48 - protein, 25 aa, Rattus sp.
PSFTIGKSVWLLWALVFNNSVPIEN

SEQ ID NO: 49 - protein, 25 aa, Rattus sp.
STFTIGKSIWLLWALVFNNSVPVEN

SEQ ID NO: 50 - protein, 25 aa, Rattus sp.
KVFSFSSALNVCYALLFGRTAAIKP

SEQ ID NO: 51 - protein, 25 aa, Rattus sp.
TVFSYSSALNLCYAILFGRTVSSKT

SEQ ID NO: 52 - DNA, 30 bp, Artificial Sequence
  syntheitc oligonucleotide
ctagcggcgcgccttaattaagtttaaacg

SEQ ID NO: 53 - DNA, 30 bp, Artificial Sequence
  syntheitc oligonucleotide
ctagcgtttaaacttaattaaggcgcgccg

(19) United States
(12) Patent Application Publication
(10) Pub. No.: US 2003/0092004 A1
(43) Pub. Date: May 15, 2003

(54) Excitatory glycine receptors and methods

(76) Inventors: Stuart A. Lipton, Rancho Santa Fe, CA (US); Dongxian Zhang, San Diego, CA (US); Jon E. Chatterton, San Diego, CA (US); Marc Awobuluyi, Boston, MA (US); Kevin A. Sevarino, Cheshire, CT (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/934,070

(22) Filed: Aug. 20, 2001

(51) Int. Cl. C12Q 1/68; C07H 21/04; C12P 21/02; C12N 5/06; C07K 14/705

(52) U.S. Cl. 435/6; 435/69.1; 435/320.1; 435/368; 530/350; 536/23.5

(57) ABSTRACT

The invention provides isolated NR3B polypeptides, functional fragments and peptides, encoding nucleic acid molecules and polynucleotides, and specific antibodies. Also provided are excitatory glycine receptors, containing either NR3B or NR3A polypeptides. Further provided are methods for detecting excitatory glycine receptor ligands, agonists and antagonists. The invention also provides related diagnostic and therapeutic methods.

This invention was made with United States Government support under grant numbers PO1 HD29587 and RO1 EY05477 awarded by the National Institutes of Health. The U.S. Government has certain rights in this invention.

### BACKGROUND OF THE INVENTION

1. Field of the Invention

This invention relates generally to the fields of neurobiology and medicine and, more specifically, to the field of ionotropic receptors. 2. Background Information

Ionotropic glutamate receptors activate ligand-gated cation channels that mediate the predominant component of excitatory neurotransmission in the central nervous system (CNS). These receptors have been classified based on their preference for the glutamate-like agonists (RS)-2-amino-3-(3-hydroxy-5-methyl-4-isoxazolyl)propionic acid (AMPA), kainate (KA), and N-methyl-D-aspartate (NMDA). All three glutamate receptor subtypes are heteromultimeric complexes, and many of the subunits that comprise them have been identified and characterized. To date, four AMPA receptor subunits (GluR1-4), five KA receptor subunits (GluR5-7, KA1, and KA2), and six NMDA receptor subunits (NR1, NR2A-2D and NR3A) have been reported.

The NMDA receptor (NMDAR) has unique properties that distinguish it from the other glutamate receptor subtypes. First, the activation of NMDAR requires the presence of dual agonists, glutamate (or NMDA) and glycine. In addition, activation of these receptors is regulated by Mg²⁺ in a voltage-dependent manner (i.e., the NMDAR is blocked at resting membrane potential and activated when depolarized). Most importantly, the NMDAR is extremely permeable to Ca²⁺, a key regulator of cell function. These unique properties allow NMDARs to play a critical role in development of the nervous system, synaptic plasticity, memory, and other physiological processes in the CNS. However, excessive stimulation of NMDARs has also been implicated in many pathological conditions including stroke, ischemia, head and spinal trauma, headache, epilepsy, neuropathic pain syndromes including diabetic neuropathy, glaucoma, depression and anxiety, drug addiction/withdrawal/tolerance, and in chronic neurodegenerative states, such as Alzheimer's disease, Huntington's disease, HIV-associated dementia, Parkinson's disease, multiple sclerosis, and amyotrophic lateral sclerosis (ALS).

The molecular cloning and functional analysis of expressed NR1, NR2A-D, and NR3A subunits, coupled with the examination of their temporal and spatial expression patterns in vivo, has led to significant advances in our understanding of NMDAR function at the molecular level. However, the identification of these six subunits alone has failed to explain the observed diversity in NMDAR function, particularly in motor neurons.

Thus, there exists a need to identify and characterize additional NMDAR subunits, and to characterize the function of additional NMDA receptors. There also exists a need to provide screening assays that identify compounds that modulate the function of additional NMDA receptors. Such compounds can be used to treat pathological conditions in which inappropriate NMDA receptor activation, or inappropriate responses to glycine or glutamate, are involved. The present invention satisfies these needs and provides related advantages as well.

### SUMMARY OF THE INVENTION

The invention provides isolated nucleic acid molecules encoding N-methyl-D-aspartate (NMDA) receptor type 3B (NR3B) polypeptides, including human, rat and mouse NR3B polypeptides.

Also provided are vectors and cells containing isolated nucleic acid molecules encoding NR3B polypeptides.

The invention also provides a method of producing an NR3B polypeptide by expressing a nucleic acid molecule encoding an NR3B polypeptide in vitro or in a cell under conditions suitable for expression of the polypeptide.

Further provided are isolated NR3B nucleic acid molecules encoding functional fragments of an NR3B polypeptide, including functional fragments that bind glycine.

The invention also provides an isolated NR3B polynucleotide containing at least 17 contiguous nucleotides from a human, rat or mouse NR3B nucleic acid molecule.

Also provided is a method for detecting a nucleic acid molecule encoding a NR3B polypeptide in a sample, by contacting the sample with one or more NR3B polynucleotides, and detecting specific hybridization to the polynucleotide, thereby detecting a nucleic acid molecule encoding an NR3B polypeptide in said sample.

The invention further provides isolated NR3B polypeptides, including human, rat and mouse NR3B polypeptides.

Also provided are functional fragments of NR3B polypeptides, including functional fragments that bind glycine.

The invention also provides isolated NR3B peptides, containing at least 8 contiguous residues of an NR3B polypeptide.

Further provided is an isolated antibody or antigen binding fragment thereof, which specifically binds an isolated NR3B polypeptide.

Also provided is a method of detecting an NR3B polypeptide in a sample, by contacting the sample with an antibody which specifically binds an NR3B polypeptide, and detecting the presence of specific binding of the antibody to the sample, thereby detecting an NR3B polypeptide in the sample.

The invention also provides methods of detecting an NR3B ligand, by contacting an NR3B polypeptide or functional fragment with one or more candidate compounds under conditions suitable for detecting binding to the polypeptide, and detecting a candidate compound that binds the polypeptide, wherein such a compound is characterized as an NR3B ligand.

Further provided is a composition containing an isolated excitatory glycine receptor. In one embodiment, the excitatory glycine receptor contains and NR3B polypeptide and an NR1 polypeptide. In another embodiment, the excitatory glycine receptor contains and NR3A polypeptide and an NR1 polypeptide. Optionally, the receptor further contains an NR2A, NR2B, NR2C or NR2D polypeptide.

The invention also provides a method of detecting an excitatory glycine receptor ligand, by contacting an excitatory glycine receptor with one or more candidate compounds under conditions suitable for detecting binding to said receptor, and detecting a candidate compound that binds said receptor, wherein such a compound is characterized as an excitatory glycine receptor ligand.

Also provided is a method of detecting an excitatory glycine receptor agonist or antagonist, by contacting an excitatory glycine receptor with one or more candidate compounds under conditions suitable for detecting receptor activation, and detecting a candidate compound that alters receptor activation, wherein such a compound is characterized as an excitatory glycine receptor agonist or antagonist.

Further provided is a method of modulating a cellular response to glycine or glutamate, by introducing a nucleic acid molecule encoding an NR3B polypeptide or functional fragment into a cell, and expressing the NR3B polypeptide or functional fragment encoded by said nucleic acid molecule in said cell, whereby expression of the polypeptide or functional fragment modulates a cellular response to glycine or glutamate.

The invention further provides a method of modulating a cellular response to glycine or glutamate, by introducing an antisense nucleic acid molecule, a ribozyme molecule or a small interfering RNA (siRNA) molecule into the cell, wherein the molecule hybridizes to an NR3B nucleic acid molecule and prevents translation of the encoded NR3B polypeptide.

### BRIEF DESCRIPTION OF THE DRAWINGS

FIG. 1 shows an alignment of the deduced amino acid sequence of the ionotropic glutamate receptor subunits from rat, designated NR1 (SEQ ID NO:14), NR2A (SEQ ID NO:15), NR3B B4 (SEQ ID NO:4) and NR3A (SEQ ID NO:16). Sequences were aligned using ClustalW and the BLOSUM series protein scoring matrix. Exact matches are boxed and shaded; conservative substitutions are boxed (no shading). Predicted signal peptide cleavage sites are indicated by vertical lines. Membrane regions (M1-M4) are indicated by horizontal lines. Asterisks indicate the positions of amino acid residues in NR1 and NR2A which have been shown to be required for glycine and glutamate binding, respectively. An arrow marks the positon of the conserved asparagine residue in NR1 and NR2A-D.

FIG. 2 shows the distribution of the NR3B subunit in the CNS by in situ hybridization. NR3B probes were labeled using isotopic (³³P)- (a, b) and non-isotopic (digoxigenin) (c, d) methods. Bar represents 6 mm for panel a, 3 mm for b, 50 mm for c, and 1 mm for d.

FIGS. 2a and 2b show that positive signals (arrows) were detected only by probes derived from antisense (AS) sequences, but not with sense (S) probes in adult rat tissue. Strong NR3B signals were observed in facial and trigeminal nuclei of the brainstem and in the ventral horn of the spinal cord.

FIG. 2c shows NR3B-positive cells viewed under high magnification (400×, top panel). These cells resemble motor neurons retrogradely labeled by injection of a fluorescent dye (granular blue) into leg muscles (bottom panel).

FIG. 2d shows the distribution of the NR3B subunit in the lumbar spinal cord of rats at different ages. NR3B signals developed postnatally, appearing as early as P2, reached a peak around P14, and remained elevated in the adult. The positive cells are large and are located in layer VIII and IX, suggesting that they are motor neurons. Arrows pointing to the labeled motor neurons are placed only on the right side of the spinal cord.

FIG. 3 shows pharmacological characterization of NR1/NR3B receptors in Xenopus oocytes. Data are representative of recordings from 1-9 oocytes in each case. Currents were recorded in 1.5 mM Ba²⁺ Ringers' solution from oocytes injected with 2 ng of NR1 cRNA and 12 ng of NR3B cRNA.

FIG. 3a shows a dose-response of glycine-evoked currents from NR1/NR3B receptors. Apparent inhibition at glycine concentrations greater than 10 μM may result from fast desensitization kinetics not readily resolved by the two-voltage electrode recording system. Inset: Significantly slower time course of NMDA-induced desensitization of NR1/NR2A currents (100 μM NMDA plus 10 μM glycine). Rebound or resensitization of NR1/NR3B receptors at high glycine concentrations upon agonist washout may result from slower clearance of glycine from the oocyte-vitelline membrane space.

FIG. 3b_{1,3} show that D-serine, an NR1/NR2 receptor co-agonist, evokes small NR1/NR3B currents alone but inhibits glycine-evoked responses in a dose-dependent fashion. FIG. 3b₂ shows that application of other NR1 glycine site agonists alone, including D-alanine, the cyclopropyl analogue ACPC or D-cycloserine (at concentrations sufficient to produce maximal or near maximal co-agonist activity at classical NMDARs) does not evoke detectable NR1/NR3B currents, but evokes NR1/NR2A currents when co-applied with 100 μM NMDA (inset). FIG. 3b₃ shows that co-application of 10 μM glycine with maximally effective doses of D-serine, D-alanine, ACPC or D- cycloserine at classical NMDARs produces relative inhibition of glycine-evoked NR/NR3B currents.

FIG. 3c shows that NMDA (100 μM) does not potentiate 10 μM glycine-evoked currents.

FIG. 3d₁₋₂ shows that kainate (100 μM) potentiates NR1/NR3B currents evoked by 5 μM glycine, while L-cysteine (50 μm), L -aspartate (100 μM) or glutamate (100 μM, not shown) have little effect or only slightly potentiate the current.

FIG. 3e₁ shows that Mg²⁺ weakly inhibits glycine-evoked NR1/NR3B currents in a dose-dependent fashion, but much less potently than receptors containing NR2 subunits. Marked rebound or resensitization occurs upon Mg²⁺ washout. Inset: Mg²⁺ inhibition of NR1/NR2A receptors (bottom bar: 100 μM NMDA plus 10 μM glycine; top bar: 100 μM NMDA, 10 μM glycine, 0.1 mM Mg²⁺).

FIG. 3e₂ shows that MK801 does not block and in fact appears to slightly potentiate glycine-evoked NR1/NR3B currents. Inset: 10 μM MK801 produces complete blockade of NR1/NR2A currents evoked by 100 μM NMDA plus 10 μM glycine.

FIG. 3e₃ shows that Memantine (12 μM) only partially blocks glycine-evoked NR1/NR3B currents but completely blocks NR1/NR2A currents evoked by 100 μM NMDA plus 10 μM glycine (inset).

FIG. 3f₁ shows that 10 μM D-aminophosphonovaleric acid (APV), a classical competitive NMDAR antagonist, has little effect on NR1/NR3B currents but blocks NR1/NR2A currents evoked by 100 μM NMDA plus 10 μM glycine (inset).

FIG. 3f₂ shows that 5,7-dichlorokynurenic acid, an NR1 glycine site antagonist, completely inhibits NR1/NR3B currents evoked by glycine, while L-strychnine, an inhibitory glycine receptor antagonist, has no effect.

FIG. 4 shows whole-cell currents recorded from oocytes injected with NR1/NR3B (1:12) cRNA.

FIG. 4a shows currents recorded at a holding potential of −60 mV in response to application of 10 μM glycine. Application of 0.5 mM Mg²⁺ had no effect, but 100 mM N-methyl-D-gluconate (NMDG) completely blocked the current. The NMDA antagonist APV (40 μM) did not block the glycine-induced current, and actually potentiated the current.

FIG. 4b shows glycine-induced currents recorded at different membrane potentials.

FIG. 4c shows currents recorded in response to a voltage ramp from −60 to +80 mV (140 mV/sec). The currents reversed around −10 mV. In the presence of NMDG the current did not reverse. No current was carried by 100 mM Ca²⁺, suggesting that channels composed of NR3B subunits are not permeable to Ca²⁺ (not shown). These findings suggest that NR1/NR3B receptor-operated channels are permeable to monovalent cations, but not appreciably to Ca²⁺.

FIG. 5 shows single-channel recordings from outside-out patches obtained from oocytes injected with NR1/NR3B (1:12) cRNA.

FIG. 5a shows current recordings at a holding potential of −60 mV. Application of glycine (10 μM) activated single-channel currents, and Mg²⁺ (0.5 mM) had no significant effect on these currents. Single-channel currents are shown at higher time resolution below. The single-channel currents display a main conductance state (2.3 pA) and a sub conductance state (0.7 pA) in the presence of 2 mM Ba ²⁺ (the zero current level is shown as a dotted line).

FIG. 5b shows all point amplitude histograms of single channel currents of NR1/NR3B receptors activated by glycine.

FIG. 5c shows single-channel currents recorded at different membrane potentials.

FIG. 5d shows single channel current-voltage relationship of the main conductance (squares) and sub conductance (circle) states.

FIG. 6 shows the effect of NMDA receptor agonists and competitive antagonists on glycine-evoked NR1/NR3A currents

FIG. 6a shows that various concentration of glycine evoked desensitizing inward currents in oocytes injected with NR1 and NR3A subunits and held at −80 mV.

FIG. 6b shows that glycine-evoked currents were effectively blocked by D-serine, which in previously described NMDA receptors is normally a co-agonist binding to the NR1 subunit.

FIG. 6c shows that glycine-evoked currents under these conditions were only slightly blocked by APV, a competitive antagonist for previously described NMDA receptors.

FIG. 6d shows that 5, 7-di-Cl-Kynurenate, an antagonist that binds to the NR1 subunit, effectively blocked the glycine-evoked current of NR1/NR3A receptors.

FIG. 6e shows that the inhibitory glycine receptor blocker, strychnine, had no effect on glycine-evoked currents of NR1/NR3A receptors.

FIG. 7 shows the effect of open-channel blockers on glycine-evoked currents of NR1/NR3A receptors.

FIG. 7a shows that Mg²⁺ displays no inhibitory effect on NR1/NR3A receptors unlike its inhibitory effect on NMDA receptors containing NR2(A-D) subunits. At low glycine levels, Mg²⁺ actually potentiates glycine-evoked currents of NR1/NR3A receptors. At higher glycine levels, Mg²⁺ potentiates peak currents without affecting the steady state current. Inset: Mg²⁺ inhibition of NR1/NR2A receptors (Top bar: 100 μM NMDA plus 10 μM glycine; bottom bar: 100 μM NMDA, 10 μM glycine, 0.1 mM Mg²⁺).

FIG. 7b shows that MK801 potentiates glycine-evoked currents at both low (2.5 μM) and high (10 μM) glycine. Currents were recorded in 1.5 mM Ba²⁺ Ringers' solution.

FIG. 8 shows the deduced amino acid sequences of rat NR3B B4 (SEQ ID NO:4) and rat NR3B A2 (SEQ ID NO:2) and their alignment with the predicted sequences of mouse NR3B (SEQ ID NO:8) and human NR3B (SEQ ID NO:6). Sequences were aligned using ClustalW and the BLOSUM series protein scoring matrix. Exact matches are boxed and shaded; conservative substitutions are boxed (no shading). Gaps (−) were inserted to maximize homology. Thick horizontal lines indicate the positions of the predicted signal peptide and membrane regions (M1-M4). Dotted horizontal lines indicate the positions of the S1 and S2 ligand binding domains.

FIG. 9 shows an alignment of regions of ionotropic glutamate receptor subunits NR1, NR2A, NR2B, NR2C, NR2D, NR3A, NR3B and GluR2. Top: Residues of the S1 and S2 regions considered to be important for glutamate binding are indicated by *. Residues of the S1 and S2 regions considered to be important for glycine binding are indicated by ˆ . Bottom: Residues of the channel lumen that are accessible from either one or both sides of the channel are boxed.

FIG. 10 shows the design of an NR3B targeting vector. The DNA fragment containing mouse NR3B exons 2-10 (˜7.6 kb) was replaced by a fragment containing the neomycin resistant (Neo^{r}) gene- (˜2 kb). The 5′- (˜3.6 kb) and 3′-(˜3.2 kb) arms used for homologous recombination are indicated by the thicker lines. The targeting DNA fragment was inserted into a pGTN29 vector.

FIG. 11 shows the predicted human NR3A cDNA sequence.

FIG. 12 shows the predicted human NR3A amino acid sequence.

### DETAILED DESCRIPTION OF THE INVENTION

The present invention relates to the cloning and characterization of a seventh NMDAR subunit, designated herein NR3B. The present invention also relates to the determination that receptors containing NR3B, or receptors containing the previously identified NMDAR subunit NR3A, display strikingly distinctive properties from all previously characterized NMDARs. The invention provides molecules and methods that can be used to prevent or ameliorate conditions in which inappropriate NMDA receptor activation, or inappropriate responses to glycine or glutamate, are involved.

The invention provides an isolated nucleic acid molecule encoding a NR3B polypeptide. As used herein, the term “NR3B polypeptide” refers to a polypeptide that retains at least one biological activity characteristic of the naturally occurring mammalian NR3B polypeptides designated herein SEQ ID NOS:2, 4, 6 or 8. As disclosed herein, an exemplary biological activity characteristic of NR3B is the ability to form a subunit of an excitatory glycine receptor. An “excitatory glycine receptor” can be characterized as a receptor that responds to micromolar concentrations of glycine with a cation current. An excitatory glycine receptor can further be characterized by exhibiting any or all of the following properties: little or no response to NMDA or glutamate; little or no response to certain NR1 glycine site agonists, such as D-alanine, ACPC or D-cycloserine; inhibition of current in response to D-serine; inhibition of current in response to 5,7-dichlorokynuric acid; lack of inhibition of current in response to L-strychnine; lack of substantial inhibitory response to Mg²⁺, MK801 or memantine; enhancement of glycine-invoked current by ≧40 μM APV; relatively Ca²⁺-impermeable.

A further exemplary biological activity characteristic of NR3B is the ability to oligomerize with an NR1 subunit, and possibly further with both an NR1 and an NR2 subunit.

Yet another exemplary biological activity characteristic of NR3B is the ability to bind glycine with high affinity. The skilled person can determine other biological activities characteristic of an NR3B polypeptide designated herein SEQ ID NOS:2, 4, 6 or 8.

Isolated nucleic acid molecules encoding NR3B polypeptides can be used, for example, as templates for the recombinant expression of NR3B subunits (the uses of which are described in more detail below); as probes to detect NR3B-encoding nucleic acid molecules in samples; in in vivo and ex vivo gene therapy applications in which modulation of NR3B expression is desired; and in other therapeutic, diagnostic, screening and research applications known to those skilled in the art.

The term “isolated,” in reference to an invention nucleic acid molecule or polypeptide is intended to mean that the molecule is substantially removed or separated from components with which it is naturally associated, or is otherwise modified by the hand of man, thereby excluding nucleic acid and polypeptide molecules as they exist in nature. An isolated molecule can be in any form, such as in a buffered solution, a suspension, a lyophilized powder, attached to a solid support (e.g. as a component of an array, or on a filter or column), or in a cell or cell extract.

The term “nucleic acid molecule,” as used herein, refers to a polynucleotide of natural or synthetic origin. A nucleic acid molecule can be single- or double-stranded genomic DNA, cDNA or RNA, and can represent a sense strand, an antisense strand, or both. Accordingly, a designated sequence identifier, unless specified otherwise, is intended to refer to the single-stranded molecule having the recited sequence, the single-stranded complement of the recited sequence, or a double stranded (or partially double-stranded) molecule in which one strand has the recited sequence.

A nucleic acid molecule can optionally include one or more non-native nucleotides, having, for example, modifications to the base, the sugar, or the phosphate portion, or having a modified phosphodiester linkage. Such modifications can be advantageous in increasing the stability of the nucleic acid molecule. Furthermore, a nucleic acid molecule can include, for example, a detectable moiety, such as a radiolabel, a fluorochrome, a ferromagnetic substance, a luminescent tag or a detectable binding agent such as biotin. Such modifications can be advantageous in applications where detection of a hybridizing nucleic acid molecule is desired.

An isolated nucleic acid molecule encoding a NR3B polypeptide can encode SEQ ID NO:6, or encode a polypeptide having at least 60% identity to SEQ ID NO:6, such as at least 70%, 80%, 85%, 90%, 95%, 97%, 99% or greater identity to SEQ ID NO:6. Identity of any two nucleic acid or amino acid sequences can be determined by those skilled in the art based, for example, on known computer alignments such as BLAST 2.0, ClustalW and the like, which can be adjusted manually, if appropriate, to insert gaps to optimize the alignment according to standard practice in the art.

An isolated nucleic acid molecule encoding a NR3B polypeptide with at least 60% identity to SEQ ID NO:6 can encode a naturally occurring or a non-naturally occurring amino acid sequence. SEQ ID NO:6 represents the predicted amino acid sequence of a naturally occurring human NR3B polypeptide.

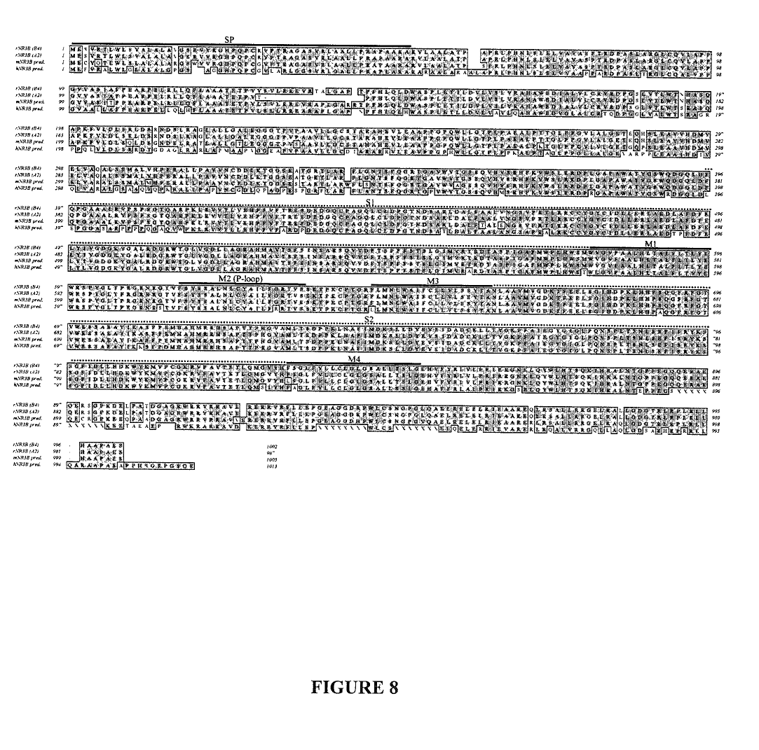

The skilled person will appreciate from the alignment shown in FIG. 8 that the C-terminus of SEQ ID NO:6 differs somewhat from the rat and mouse orthologs. Based on these observed differences, it is contemplated that a naturally occurring human NR3B polypeptide can contain additional sequence corresponding to one or more of the gaps where SEQ ID NO:6 does not apparently align with SEQ ID NOS: 2, 4 and 8, or no sequence in these positions.

For example, it is contemplated that a naturally occurring human NR3B polypeptide can contain several additional amino acids (e.g. 1-20 additional amino acids, such as 11 additional amino acids), or no amino acids, between the sequence “PPEGS” and “KEETA” of SEQ ID NO:6. Such additional sequence can be identical to, substantially similar to, or different from, the sequence QQERAEQERSGP (portion of SEQ ID NO:4) or the sequence QQERAEQECRGP (portion of SEQ ID NO:8). Likewise, it is contemplated that a naturally occurring human NR3B polypeptide can contain several additional amino acids (e.g. 1-20 additional amino acids, such as 8 additional amino acids), or no amino acids, between the sequence “FLLEP” and “WLCS” of SEQ ID NO:6. Such additional sequence can be identical to, substantially similar to, or different from, the sequence GEAGGDRP (portion of SEQ ID NO:4) or the sequence GEAGGDHP (portion of SEQ ID NO:8).

Likewise, it is contemplated that a naturally occurring human NR3B polypeptide can contain several additional amino acids (e.g. 1-20 additional amino acids, such as 7 additional amino acids), or no amino acids, between the sequence “WLCS” and “ELQEL” of SEQ ID NO:6. Such additional sequence can be identical to, substantially similar to, or different from, the sequence NGPGLQA (portion of SEQ ID NO:4) or the sequence NGPGVQA (portion of SEQ ID NO:8). Furthermore, it is contemplated that a naturally occurring human NR3B polypeptide does not contain the residues in SEQ ID NO:6 that extend beyond the corresponding residues from the C-terminus of SEQ ID NO:4 and 8, such as the sequence PPHSGRPGSQE (portion of SEQ ID NO:6).

A human NR3B polypeptide can contain C-terminal amino acid sequences that are not present in a sequence submitted to GenBank and annotated as a hypothetical protein most similar to rat ionotropic gluatmate receptor (L34938) with an ill-defined C-terminus (GenBank entry AC004528 and AAC12680; SEQ ID NOS:9 and 10). In particular, a human NR3B polypeptide can contain any or all of the C-terminal portion of SEQ ID NO:6 not also present in SEQ ID NO:10, such as the sequence XXXXXXXXXXXXXWKRARRAVDKERRVRFLLEPXXXXXXXXWLCSXXXXXXXELQEL ERRIEVARERLRQALVRRGQLLAQLGDSARHRPRRLLQARAAPAEAPPHSGRPGSQE where X can be any amino acid.

SEQ ID NOS:4 and 2 represent the predicted amino acid sequence of a naturally occurring rat NR3B polypeptide. SEQ ID NO:8 represents the predicted amino acid sequence of a naturally occurring mouse NR3B polypeptide.

An isolated nucleic acid molecule encoding SEQ ID NO:6 can have the nucleotide sequence designated SEQ ID NO:5, which represents a naturally occurring human NR3B cDNA sequence. The skilled person understands, however, that due to the degeneracy of the genetic code, SEQ ID NO:6 can also be encoded by a nucleotide sequence that differs from SEQ ID NO:5 at one or more codons.

Likewise, isolated nucleic acid molecules encoding SEQ ID NOS:4, 2 or 8 can have the nucleotide sequences designated SEQ ID NOS:3, 1 or 7, or degenerate variants thereof.

As shown in the alignment in FIG. 8, SEQ ID NOS:2, 4, 6 and 8 are highly homologous over their entire lengths. Because of this high degree of identity of NR3B polypeptides across these three mammalian species, it is expected that other naturally occurring mammalian NR3B polypeptides, such as NR3B polypeptides from non-human primates, mouse, rat, rabbit, bovine, porcine, ovine, canine or feline species, as well as naturally occurring NR3B polypeptides from other vertebrates, including fish, birds, reptiles and amphibians (e.g. Xenopus) will also exhibit a high degree of identity across their lengths with SEQ ID NO:6.

Using knowledge of the human, rat or mouse NR3B-encoding nucleic acid sequences and polypeptides disclosed herein, those skilled in the art can readily clone NR3B-encoding nucleic acids from other mammalian or vertebrate species using conventional cDNA or expression library screening methods, or using the polymerase chain reaction (PCR). Additionally, using knowledge of the human, rat or mouse NR3B-encoding nucleic acid sequences and polypeptides disclosed herein, those skilled in the art can readily determine cDNA and coding sequences form other species from an analysis of ESTs and genomic sequences present in available databases.

In contrast, SEQ ID NO:4 exhibits about 47% identity to rat NR3A, with much lower identity to other NMDA receptor subunits (i.e. 19.7% identity to rat NR1; 18.6% identity to rat NR2A). Therefore, the skilled person can readily distinguish an NR3B polypeptide from related receptor subunits based on sequence similarity.

For certain applications, an isolated nucleic acid molecule encoding an NR3B polypeptide need not encode the naturally occurring signal peptide sequence, which is cleaved in the mature polypeptide. The predicted signal peptide sequences of rat (SEQ ID NOS:2 and 4), human (SEQ ID NO:6) and mouse (SEQ ID NO:8) NR3B polypeptides are shown by overlining and the designation “SP” in FIG. 8. Accordingly, in one embodiment, an isolated nucleic acid molecule can encode an NR3B polypeptide in which some or all or of amino acids 1-51 or 1-53 of SEQ ID NOS:2, 4, 6 or 8 are not present. The skilled person can readily determine the boundaries of the signal peptide sequence from NR3B polypeptides and, if desired, replace these residues with another signal or sorting sequence.

An isolated nucleic acid molecule encoding an NR3B polypeptide can also be a splice variant form that differs from another form by one or more exons, thereby encoding a NR3B polypeptide that differs from another NR3B polypeptide by an insertion or deletion of one or more residues at one or more places in the polypeptide. NR3B splice variants can be expressed in a tissue or developmental stage-specific manner. Rat NR3B A2 (SEQ ID NO:2) and rat NR3B B4 (SEQ ID NO:4) are examples of splice variant forms that differ by containing (SEQ ID NO:4) or not containing (SEQ ID NO:2) the sequence VSVLRREVRTALGAP (portion of SEQ ID NO:4). An exemplary splice variant of a human NR3B differs from SEQ ID NO:6 by not containing the sequence LSLLRREARAPLGAP (portion of SEQ ID NO:6) or by not containing the sequence LSLLRREARAPLGAPN (portion of SEQ ID NO:6). An exemplary splice variant of a mouse NR3B differs from SEQ ID NO:8 by not containing the sequence LSVLRREVRAPLGAR (portion of SEQ ID NO:8) or by not containing the sequence LSVLRREVRAPLGARR (portion of SEQ ID NO:8). The skilled person can readily determine additional splice variants of these and other NR3B polypeptides.

An isolated nucleic acid molecule encoding an NR3B polypeptide can also have one or more minor modifications to the naturally occurring sequence, such as one or more substitutions additions or deletions. Such modifications can be advantageous, for example, in enhancing the stability, bioavailability, bioactivity or immunogenicity of the polypeptide, or to facilitate its purification.

Substitutions to an NR3B amino acid sequence can either be conservative or non-conservative. Conservative amino acid substitutions include, but are not limited to, substitution of an apolar amino acid with another apolar amino acid (such as replacement of leucine with an isoleucine, valine, alanine, proline, tryptophan, phenylalanine or methionine); substitution of a charged amino acid with a similarly charged amino acid (such as replacement of a glutamic acid with an aspartic acid, or replacement of an arginine with a lysine or histidine); substitution of an uncharged polar amino acid with another uncharged polar amino acid (such as replacement of a serine with a glycine, threonine, tyrosine, cysteine, asparagine or glutamine); or substitution of a residue with a different functional group with a residue of similar size and shape (such as replacement of a serine with an alanine; an arginine with a methionine; or a tyrosine with a phenylalanine).

Additions to an NR3B amino acid sequence include, but are not limited to, the addition of “tag” sequences, which are conveniently added at the N- or C-termini, after the signal peptide, or within extracellular or intracellular loops. Such tag sequence include, for example, epitope tags, histidine tags, glutathione-S-transferase (GST), fluorescent proteins (e.g. Enhanced Green Fluorescent Protein (EGFP)) and the like. Such additional sequences can be used, for example, to facilitate expression, purification or characterization of an NR3B polypeptide.

Deletions to an NR3B amino acid sequence include, but are not limited to, deletion of signal peptide residues, and deletion of residues at the N- or C- termini that are not critical for function. Deleted sequences can optionally be replaced by tag sequences or fusion sequences, as described previously.

Modifications to an encoded NR3B amino acid sequence, such as modifications to any of SEQ ID NOS:2, 4, 6 or 8, can be randomly generated, such as by random insertions, deletions or substitutions of nucleotides in a nucleic acid molecule encoding the polypeptide. Alternatively, modifications can be directed, such as by site-directed mutagenesis of a nucleic acid molecule encoding the polypeptide.

Guidance in modifying the sequence of an NR3B polypeptide while retaining biological activity can be provided by the alignment of the sequence of the NR3B orthologs from human, rat and mouse shown in FIG. 8. It is well known in the art that evolutionarily conserved amino acid residues are more likely to be important for maintaining biological activity than less well-conserved residues. Thus, it would be expected that substituting a residue that is highly conserved among NR3B polypeptides across species with a non-conserved residue may be deleterious, whereas making the same substitution at a residue which varies among species would likely not have a significant effect on biological activity.

Additionally, guidance in modifying amino acid residues of an NR3B polypeptide while retaining a desired biological activity can be provided by structure-function studies of known NMDA receptor subunits, which share an overall transmembrane topology and domain structure with NR3B. By analogy to other subunits, the ligand binding domain of NR3B is predicted to be formed by the extracellular S1 domain before the first membrane spanning region (M1) and by the extracellular S2 domain between membrane spanning regions M3 and M4 (FIG. 8). The second membrane domain (M2, or P-loop) is predicted to line the ion channel pore (see FIG. 8). Meddows et al., J. Biol. Chem. 276:18795-18803 (2001) have also determined that retention of the N-terminal residues of the NMDA receptor subunit NR1 (i.e. amino acids 1-380 of NR1) is important for subunit oligomerization, whereas the M4 domain and C-terminal residues (i.e. amino acids 811-938 of NR1) are dispensible for oligomerization but required for functional channel formation. The skilled person could apply this knowledge to predict the effect of various modifications within the above-described structural and functional domains on NR3B biological activity.

Computer programs well known in the art can also provide guidance in predicting which amino acid residues can be modified without abolishing a topological or functional feature of an NR3B polypeptide.

The invention also provides an isolated nucleic acid molecule that encodes a functional fragment of an NR3B polypeptide. As used herein, the term “functional fragment” refers to a portion of a full-length NR3B polypeptide that retains at least one biological activity characteristic of the full-length polypeptide. A functional fragment can contain, for example, at least about 6, 8, 10, 12, 15, 20, 25, 30, 35, 40, 50, 60, 70, 80, 90, 100, 110, 125, 150, 200, 250, 300, 400, 500, 600, 700, 800, 900, 950 or more amino acids of an NR3B polypeptide.

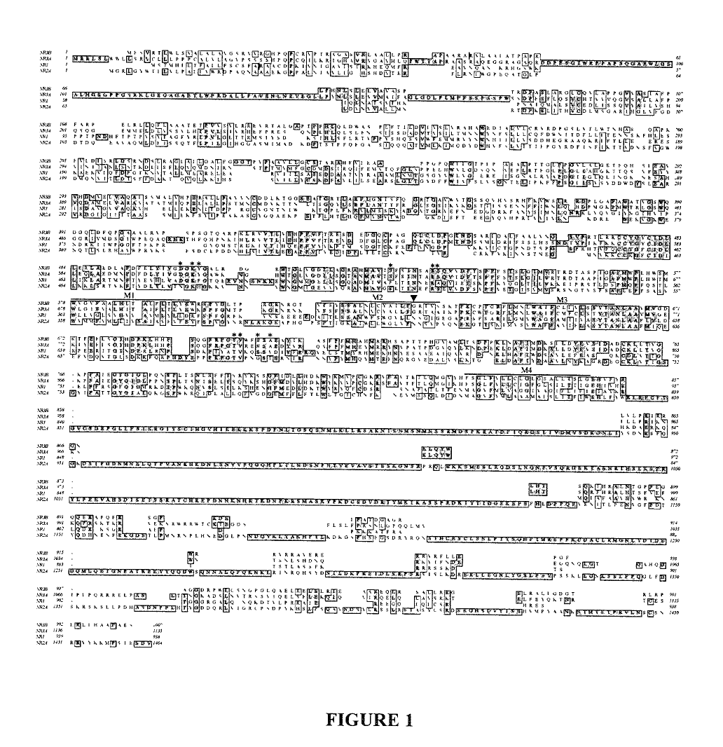

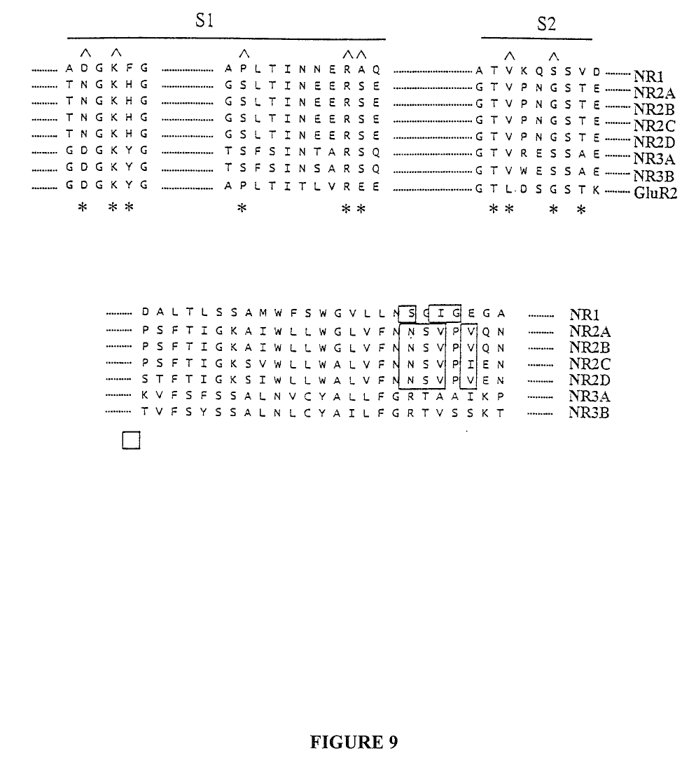

For example, a functional fragment of an NR3B polypeptide can retain the ability to bind glycine. As shown in FIGS. 1 and 9, the residues of the S1 and S2 regions considered to be important in binding glycine are known. Thus, a functional fragment can contain all or part of the S1 and/or S2 domains of rat, human or mouse NR3B (see FIG. 8), and optionally further contain the naturally occurring NR3B intervening sequence, such as membrane regions M1-M3.

An exemplary NR3B functional fragment that binds glycine can contain SEQ ID NO:27 and/or SEQ ID NO:35 and/or SEQ ID NO:43. Advantageously, a chimeric polypeptide containing all or a portion of a different NMDA receptor subunit (e.g. NR1, NR2A-D or NR3A), with the glycine binding domain (e.g. the S1 and/or S2 regions; or SEQ ID NO:27 and/or SEQ ID NO:35 and/or SEQ ID NO:43) of NR3B replacing the corresponding region of the NMDA receptor subunit, can be constructed. Likewise, a chimeric polypeptide containing all or a portion of an NMDA receptor subunit (e.g. NR1, NR2A-D or NR3B), with the glycine binding domain (e.g. the S1 and/or S2 regions; or SEQ ID NO:26 and/or SEQ ID NO:34 and/or SEQ ID NO:42) of NR3A replacing the corresponding region of the NMDA receptor subunit, can be constructed. Such a functional fragment, or a chimeric polypeptide containing such a fragment, can be used, for example, in screening applications described further below to detect excitatory glycine receptor ligands, agonists and antagonists. Additionally, such a functional fragment can be used in therapeutic applications in which it is desirable to compete with an endogenous receptor for binding to agonist. Methods for making and testing chimeric glutamate receptor polypeptides are described, for example, in Villmann et al., Eur. J. Neurosci. 11:1765-1778 (1999).

A functional fragment of NR3B can also retain the ability to oligomerize with other NMDA receptor subunits, such as NR1, and optionally NR2. By analogy to NR1, such a fragment can retain all or most of the N-terminal region before the S1 domain, which is predicted to be important for oligomerization (see Meddows et al., supra (2001)).

A further exemplary functional fragment of NR3B can retain the ability to insert into the membrane or form a channel pore by retaining some or all of the membrane regions (M1-M4). Such fragments can be used, for example, to compete with or disrupt the structure of the naturally occurring NR3B.

Another exemplary functional fragment of NR3B can retain the ability to interact with intracellular proteins, such as effector proteins, by retaining some or all of the intracellular region C-terminal to M4. Such fragments can be used, for example, in binding assays to identify polypeptides that interact with NR3B, which can then themselves be used as targets in screening assays; and also can be used to compete with naturally occurring NR3B for binding to effector polypeptides.

Accordingly, the invention provides an isolated nucleic acid molecule that encodes an NR3B functional fragment that contains the extracellular domain of an NR3B polyepeptide N-terminal to the S1 domain (with or without the signal peptide), and/or the S1 domain, and/or the M1 domain, and/or the M2 domain, and/or the and/or the M3 domain, and/or the S2 domain, and/or the M4 domain, and/or the intracellular domain C-terminal to the M4 domain. The boundaries of these domains for several mammalian NR3B polypeptides are shown in FIG. 8. The skilled person can determine appropriate functional fragments of an NR3B polypeptide for use in a particular application.

The biological activities of NR3B polypeptides and functional fragments can be determined or confirmed by methods known in the art and described further in the Examples. For example, the ability of an NR3B polypeptide or functional fragment to act as a subunit of an excitatory glycine receptor can be tested by recombinantly expressing an NR3B polypeptide in an appropriate cell (e.g. a Xenopus oocyte or mammalian cell) in the presence of a suitable amount of another endogenous or exogenous NMDAR subunit (e.g. an NR1 subunit, an NR2 subunit, or an NR3A subunit, or any combination thereof that includes an NR1 subunit), and detecting currents evoked in a dose-dependent fashion by addition of glycine. Other suitable methods for detecting the ability of an NR3B polypeptide to act as a subunit of an excitatory glycine receptor are known in the art and described further below with respect to screening assays.

The ability of an NR3B polypeptide or functional fragment to oligomerize with an NR1 and/or an NR2 and/or an NR3A polypeptide can be assayed, for example, by a functional assay to measure excitatory ionic responses of an NR3B/NR1 receptor to glycine, as described above, or alternatively by co-expressing the polypeptides and detecting NR3B/NR1 polypeptide association. Various assays for detecting polypeptide associations are well known in the art and include, for example, co-immunoprecipitation assays (see, for example, Meddows et al., supra (2001)), two-hybrid assays, GST pull-down assays, protein chip proteomic array analysis (e.g. ProteinChip™ System from Ciphergen Biosystems, which can be used in tandem with mass spectrometry analysis for sequence or structure determination) and the like, using an NR3B polypeptide or functional fragment.

The ability of an NR3B polypeptide or functional fragment to bind glycine can be also detected by a functional assay to measure excitatory ionic responses of an NR3B/NR1 receptor to glycine, as described above, or alternatively by a ligand binding assay. Various direct and competitive ligand binding assays, such as those described above with respect to oligomerization, and assays described below with respect to screening, are well known in the art and can be used to determine the ability of an NR3B polypeptide or functional fragment to bind glycine.

Further provided are isolated polynucleotides containing at least 17 contiguous nucleotides of an invention NR3B nucleic acid molecule or of its complement. An isolated polynucleotide can thus contain at least 18, 19, 20, 22, or at least 25 contiguous nucleotides, such as at least 30, 40, 50, 60, 70, 80, 90, 100, 125, 150, 175, 200, 225, 250, 275, 300, 350, 400, 500, 600, 700, 800, 1000, 1500, 2000, 2500, 3000, 3500 or more contiguous nucleotides from the reference nucleotide sequence, up to the full length sequence. An invention polynucleotide can be single or double stranded, represent the sense or antisense strand, and contain either coding or non-coding sequence or both. An invention polynucleotide can, but need not, encode a biologically active polypeptide and can, but need not, be inserted into a vector.

In one embodiment, the isolated polynucleotide comprises at least 17 contiguous nucleotides of any of SEQ ID NOS:1, 3, 5 or 7 or the complement thereof. Such polynucleotides are of sufficient length and complexity to be able to specifically hybridize to an NR3B-encoding nucleic acid molecule under highly stringent hybridization conditions. Therefore, the invention polynucleotides can advantageously be used, for example, as probes to detect the presence, abundance or fidelity of NR3B-encoding nucleic acid molecules in a sample; as NR3B-specific sequencing or PCR primers; as antisense, RNA interference or ribozyme reagents for use in ex vivo or in vivo gene therapy applications to block expression of NR3B in a cell, as described in more detail below; or in other applications known to those skilled in the art in which hybridization to an NR3B-encoding nucleic acid molecule is desirable. In certain applications, polynucleotides that distinguish a splice variant form of an NR3B receptor are useful, such as polynucleotides containing the region of the rat NR3B B4 form not present in the NR3B A2 form.

Specific hybridization refers to the ability of a nucleic acid molecule to hybridize to the reference nucleic acid molecule without hybridization under the same conditions with nucleic acid molecules that are not the reference molecule, such as a nucleic acid molecule encoding another NMDA receptor subunit. Moderately stringent hybridization conditions are conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 50°. Highly stringent conditions are conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 65° C. Other suitable moderately stringent and highly stringent hybridization buffers and conditions are well known to those of skill in the art and are described, for example, in Sambrook et al., Molecular Cloning: A Laboratory Manual, 3rd ed., Cold Spring Harbor Press, Plainview, N.Y. (2001) and in Ausubel et al. (Current Protocols in Molecular Biology (Supplement 47), John Wiley & Sons, New York (1999)).

In one embodiment, the invention provides a primer pair containing two isolated polynucleotides as set forth above. The primer pair can be used, for example, to amplify an NR3B-encoding nucleic acid molecule by the polymerase chain reaction (PCR). A suitable primer pair can contain an isolated polynucleotide containing at least 17 contiguous nucleotides of the sense strand of an invention NR3B nucleic acid molecule, and an isolated polynucleotide containing at least 17 contiguous nucleotides of the antisense strand of an invention NR3B nucleic acid molecule. The skilled person can determine an appropriate primer length and sequence composition for the intended application.

NR3B nucleic acid molecules (including nucleic acid molecules encoding NR3B polypeptides, functional fragments thereof and polynucleotides, as described above) can optionally contain exogenous nucleotide sequences including, for example, sequences that facilitate identification or purification of the molecule, and sequences that facilitate cloning, such as restriction endonuclease recognition sites.

NR3B nucleic acid molecules can be produced or isolated by methods known in the art. The method chosen will depend on the type of nucleic acid molecule one intends to isolate. Those skilled in the art, based on knowledge of the nucleotide sequences disclosed herein, can readily isolate NR3B nucleic acid molecules as genomic DNA, as full-length cDNA or desired fragments therefrom, or as full-length mRNA or cRNA or desired fragments therefrom, by methods known in the art.

It will be appreciated that an invention NR3B polypeptide, functional fragment or peptide does not consist of the exact sequence of an amino acid sequence set forth in a publically available database, or of the exact amino acid sequence of a translated product of a nucleic acid molecule set forth in a publically available database. Likewise, an invention nucleic acid molecule encoding a NR3B polypeptide or functional fragment, or an NR3B polynucleotide, does not consist of the exact sequence of a nucleotide sequence set forth in publically available databases, including but not limited to Expressed Sequence Tags (ESTs), Sequence Tagged Sites (STSs) and genomic fragments deposited in public databases such as the GenBank nr, dbest, dbsts and gss databases.

Specifically excluded from the invention polypeptides and nucleic acid molecules are molecules having the exact sequence of any of the following: the human EST sequence designated SEQ ID NO:13 (GenBank Accession No. AL359933); fragments of human chromosome 19 genomic sequences (e.g. GenBank Accession No. AC004528), such as the predicted cDNA sequence designated SEQ ID NO:9 which encodes a protein designated as a hypothetical human protein most similar to rat ionotropic gluatmate receptor, and the encoded polypeptide, SEQ ID NO:10 (GenBank Accession No. AAC12680); the mouse EST sequence designated SEQ ID NO:11 (GenBank Accession No. BC005494) and its predicted encoded polypeptide designated SEQ ID NO:12 (GenBank Accession No. AAH05494.1); and deposited fragments of mouse chromosome 10 genomic sequences (e.g. GenBank Accession No. AC087114).

Since one of skill in the art will realize that the above-recited excluded sequences may be revised in the database at a later date, it is intended that the above-recited sequences are excluded as they stand on the priority date of this application.

Isolated NR3B nucleic acid molecules can be prepared or isolated by methods well known in the art. The method chosen will depend on factors such as the type and size of the nucleic acid molecule; whether or not it encodes a biologically active polypeptide; and the source of the nucleic acid molecule. Such methods are described, for example, in Sambrook et al., supra (2001) and in Ausubel et al., supra (1999).

One useful method for producing an isolated NR3B nucleic acid molecule involves amplification of the nucleic acid molecule using the polymerase chain reaction (PCR) and specific primers and, optionally, purification of the resulting product by gel electrophoresis. Either PCR or reverse-transcription PCR (RT-PCR) can be used to produce a nucleic acid molecule having any desired nucleotide boundaries. Desired modifications to the nucleic acid sequence can also be conveniently introduced by choosing an appropriate primer with one or more additions, deletions or substitutions. Such nucleic acid molecules can be amplified exponentially starting from as little as a single gene or mRNA copy, from any cell, tissue or species of interest.

An isolated NR3B nucleic acid molecule can also be prepared by screening a library, such as a genomic library, cDNA library or expression library, with a detectable NR3B nucleic acid molecule or antibody. Human libraries, and libraries from a large variety of other species, are commercially available or can be produced from species or cells of interest. The library clones identified as containing NR3B nucleic acid molecules can be isolated, subcloned or sequenced by routine methods. From an initially identified fragment, nucleic acid molecules encoding full-length polypeptides can be obtained, if desired, by a variety of methods well-known in the art, such as 5′ or 3′ RACE.

Furthermore, an isolated NR3B nucleic acid molecule can be produced by synthetic means. For example, a single strand of a nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as probes and primers, and nucleic acid molecules containing modified nucleotides or linkages.

The invention also provides a vector containing an isolated NR3B nucleic acid molecule. The vectors of the invention are useful, for example, for subcloning and amplifying NR3B nucleic acid molecules, and for recombinantly expressing NR3B polypeptides and functional fragments thereof. A vector of the invention can include a variety of elements useful for cloning and/or expression of the encoded nucleic acid molecule in the desired host cell, such as promoter and/or enhancer sequences, which can provide for constitutive, inducible or cell-specific RNA transcription; transcription termination and RNA processing signals, including polyadenylation signals, which provide for stability of a transcribed mRNA sequence; an origin of replication, which allows for proper episomal replication; selectable marker genes, such as a neomycin or hygromycin resistance gene, useful for selecting stable or transient transfectants in mammalian cells, or an ampicillin resistance gene, useful for selecting transformants in prokaryotic cells; and versatile multiple cloning sites for inserting nucleic acid molecules of interest.

Cloning vectors of the invention include, for example, viral vectors such as a bacteriophage, a baculovirus or a retrovirus; cosmids or plasmids; and, particularly for cloning large nucleic acid molecules, bacterial artificial chromosome vectors (BACs) and yeast artificial chromosome vectors (YACs). Such vectors are commercially available, and their uses are well known in the art.

If it is desired to express NR3B RNA transcripts or polypeptides, an invention nucleic acid molecule can be operatively linked to a promoter of RNA transcription. The term “operatively linked,” as used herein, is intended to mean that the nucleic acid molecule is positioned with respect to the endogenous promoter, or heterologous promoter, in such a manner that the promoter will direct the transcription of RNA using the nucleic acid molecule as a template. Methods for operatively linking a nucleic acid to a desired promoter are well known in the art and include, for example, cloning the nucleic acid into a vector containing the desired promoter, or appending the promoter to a nucleic acid sequence using PCR.

Thus, an invention nucleic acid molecule operatively linked to a promoter of RNA transcription can be used to express NR3B transcripts and polypeptides in a desired host cell, or in an in vitro system, such as an extract or lysate that supports transcription and translation.

Contemplated promoters and expression vectors provide for expression in bacterial cells, yeast cells, insect cells, amphibian cells, mammalian cells (including human, non-human primate and rodent cells) and other vertebrate cells. A variety of promoters and expression vectors suitable for such purposes are commercially available, and can be further modified, if desired, to include appropriate regulatory elements to provide for the desired level of expression or replication in the host cell.

For use in the gene therapy applications described further below, an invention nucleic acid molecule can be incorporated into suitable gene therapy vector, such as a viral vector or plasmid. Viral based vectors are advantageous in being able to introduce relatively high levels of a heterologous nucleic acid into a variety of cells, including nondividing cells.

Suitable viral vectors for gene therapy applications are well known in the art, and include, for example, Herpes simplex virus vectors (U.S. Pat. No. 5,501,979), Vaccinia virus vectors (U.S. Pat. No. 5,506,138), Cytomegalovirus vectors (U.S. Pat. No. 5,561,063), Modified Moloney murine leukemia virus vectors (U.S. Pat. No. 5,693,508), adenovirus vectors (U.S. Pat. Nos. 5,700,470 and 5,731,172), adeno-associated virus vectors (U.S. Pat. No. 5,604,090), constitutive and regulatable retrovirus vectors (U.S. Pat. Nos. 4,405,712; 4,650,764 and 5,739,018, 5,646,013, 5,624,820, 5,693,508 and 5,674,703), papilloma virus vectors (U.S. Pat. Nos. 5,674,703 and 5,719,054), lentiviral vectors (Kafri et al., Mol. Ther. 1:516-521 (2000), and the like. For targeting neural cells in the treatment of neuronal diseases, adenoviral vectors, Herpes simplex virus vectors and lentiviral vectors are particularly useful.

The invention also provides a cell containing an isolated NR3B nucleic acid molecule. Such a cell need not express a recombinant NR3B polypeptide or fragment for use in cloning procedures. However, a cell can optionally express an NR3B polypeptide or functional fragment encoded by the nucleic acid molecule. Such cells can be used in a variety of applications including, for example, screening for agonists, antagonists and ligands of excitatory glycine receptors, as described further below; as a source to isolate recombinantly expressed NR3B polypeptides; for identifying additional cellular molecules, such as additional receptor subunits or intracellular proteins that associate with NR3B; and in other applications known to those skilled in the art.

Optionally, a cell that recombinantly expresses an NR3B polypeptide can further endogenously or recombinantly express at least one other NMDA subunit, such as an NR1 subunit. As disclosed herein, co-expression of an NR3B and an NR1 polypeptide results in the formation of excitatory glycine receptors. NR1 polypeptides and encoding nucleic acid molecules from various species are known in the art, and include the naturally occurring NR1 polypeptides from human, rat, mouse, duck, fish and Xenopus having the nucleotide and predicted amino acid sequences set forth in Table 1:

TABLE 1

| SUBUNIT | ACCESSION NUMBER |
|---|---|
| Human NR1 | D13515 |
| Human NR1-1 | L13266 |
| Human NR1-2 | L13267 |
| Human NR1-3 | L13268 |
| Human NR1-3b | AF015730 |
| Human NR1-4b | AF015731 |
| Rat NR1 | U11418 |
| Rat NR1 | X63255 |
| Rat NR1-2b | U08264 |
| Rat NR1-3a | U08265 |
| Rat NR1-3b | U08266 |
| Rat NR1-4a | U08267 |
| Rat NR1-4b | U08268 |
| Mouse NR1 | D10028 |
| Duck NR1 | D83352 |
| Fish NR1 | AF060557 |
| Rat NR1-1a | U08261 |
| Rat NR1-2a | U08262 |
| Rat NR1-1b | U08263 |
| Xenopus NR1 | X94081 |

The skilled person will appreciate that for use in the methods described herein, a co-expressed NR1 polypeptide can have modifications to the naturally occurring sequence, or be a functional fragment of the naturally occurring sequence, so long as the desired NR1 biological activity is retained. An exemplary modification of a naturally occurring NR1 polypeptide that does not affect biological activity is the addition of an epitope tag to facilitate identification in procedures such as immunolocalization and immunoprecipitation. NR1 biological activities include, for example, the ability to oligomerize with other NMDA subunits, including NR2A-D, NR3B and NR3A; the ability to bind glycine; the ability to form excitatory glycine receptors in association with either NR3B or NR3A; and the ability to form NMDA- and glycine-responsive receptors in association with NR2 subunits. As described above with respect to NR3B polypeptides and functional fragments, the skilled person can readily make NR1 molecules with sequences that differ from the naturally occurring sequence and test such molecules to confirm that a desired NR1 biological activity is retained.

Exemplary host cells that can be used to recombinantly express receptor polypeptides and fragments include mammalian primary cells; established mammalian cell lines, such as COS, CHO, HeLa, NIH3T3, HEK 293-T and PC12 cells; amphibian cells, such as Xenopus embryos and oocytes; and other vertebrate cells. Exemplary host cells also include insect cells (e.g. Drosophila), yeast cells (e.g. S. cerevisiae, S. pombe, or Pichia pastoris) and prokaryotic cells (e.g. E. coli).

Also provided are extracts of recombinant cells that express NR3B at the cell membrane, wherein the extract contains the cell membrane. Advantageously, NR3B is expected to retain a biologically active conformation in a membrane extract, but is partially purified away from other cellular components that may be undesirable for certain applications. Cell membrane extracts can be prepared by methods known in the art (see, for example Das et al., Nature 393:377-381 (1998)) and can be used in many of the screening assays described herein.

Methods for introducing a recombinant nucleic acid molecule into a host cell are well known in the art. The choice of method will depend on the host cell, the type of nucleic acid molecule, and the intended application of the host cell. Suitable methods include, for example, various methods of transfection such as calcium phosphate, DEAE-dextran and lipofection methods; viral transduction; electroporation; and microinjection.

Animal model systems can be useful for elucidating normal and pathological functions of NR3B polypeptides, and for determining the efficacy and safety of potential therapeutic compounds that target excitatory glycine receptors. Accordingly, the invention also provides a transgenic non-human animal that contains an NR3B nucleic acid molecule. Such transgenic animals can express a nucleic acid molecule encoding an invention NR3B polypeptide or functional fragment, including a functional fragment that competes with or inhibits the function of the naturally occurring NR3B, as described previously. Alternatively, transgenic animals can express an invention NR3B polynucleotide that prevents effective translation of the naturally occurring NR3B polypeptide, such as an antisense, ribozyme, RNAi or similar construct. By employing suitable inducible and/or tissue specific regulatory elements, NR3B expression or activity in the transgenic animal can be restricted to specific cell types, developmental stages, or induction conditions.

Any of a variety of methods known in the art can be used to introduce a desired transgene into animals to produce the founder lines of transgenic animals (see, for example, Hogan et al., Manipulating the Mouse Embryo: A Laboratory Manual, second ed., Cold Spring Harbor Laboratory (1994), U.S. Pat. Nos. 5,602,299; 5,175,384; 6,066,778; and 6,037,521). Such techniques include, but are not limited to, pronuclear microinjection (U.S. Pat. No. 4,873,191); retrovirus mediated gene transfer into germ lines (Van der Putten et al., Proc. Natl. Acad. Sci. USA 82:6148-6152 (1985)); gene targeting in embryonic stem cells (Thompson et al., Cell 56:313-321 (1989)); electroporation of embryos (Lo, Mol Cell. Biol. 3:1803-1814 (1983)); and sperm-mediated gene transfer (Lavitrano et al., Cell 57:717-723 (1989)). Once the founder animals are produced, they can be bred to produce colonies of the particular animal by methods known in the art.

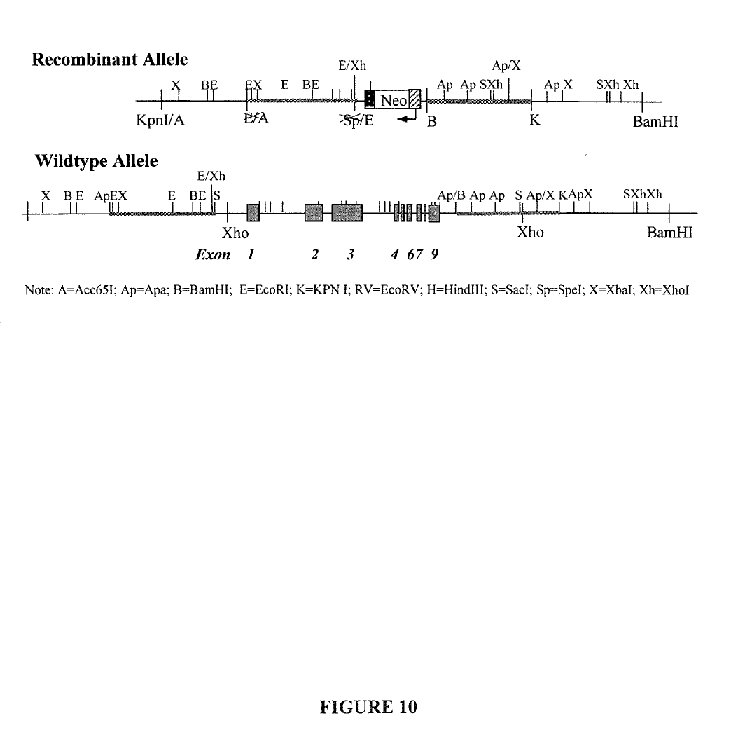

In another embodiment, the invention provides NR3B-deficient non-human animals, or NR3B “knock-out” animals. Methods of deleting all or a portion of a gene so as to alter or prevent expression of the naturally occurring polypeptide are well known in the art. Gene knockout by homologous recombination is described, for example, in Capecchi et al., Science 244:1288 (1989), and in U.S. Pat. Nos. 5,616,491, 5,750,826, and 5,981,830. Methods of making and using an NR3A knockout mouse are described in Das et al., Nature 393:377-381 (1998). Analogous targeting vectors and methods are expected to be useful in generating NR3B knockout animals. FIG. 10 describes a targeting construct suitable for use in generating an NR3B knockout mouse.

The invention also provides a method for detecting a nucleic acid molecule encoding an NR3B polypeptide in a sample. Because of the critical role NMDA receptors play in neurologic disorders, the method can be used, for example, to diagnose or prognose a pathological condition mediated, in part, by altered expression, abundance or integrity of an NR3B nucleic acid molecule. Such conditions include, for example, acute neurologic conditions and chronic neurodegenerative diseases described further below. The detection method can also be used to identify additional regions of the nervous system in which NR3B is normally or pathologically expressed. Such information can be valuable in determining additional diagnostic and therapeutic applications for the invention molecules and methods described herein. Furthermore, the detection method can also be used to identify additional naturally occurring splice variants of NR3B receptors, and NR3B-encoding nucleic acid molecules from other species of interest, such as veterinary and laboratory animals.

In one embodiment, the detection method is practiced by contacting a sample with one or more NR3B polynucleotides and detecting specific hybridization to the polynucleotide. Suitable hybridization conditions for detecting specific hybridization will depend on the detection format, and are well known in the art. Exemplary conditions useful for filter-based assays have been described previously. Example II provides exemplary conditions suitable for in situ hybridization assays. Suitable conditions for PCR-based detection methods are also well known in the art and described, for example, in Sambrook et al., supra (2001) and in Ausubel et al., supra (1999).

As used herein, the term “sample” is intended to mean any biological fluid, cell, tissue, organ or portion thereof that contains or potentially contains an NR3B nucleic acid molecule or polypeptide. For example, a sample can be a histologic section of a specimen obtained by biopsy, or cells that are placed in or adapted to tissue culture. A sample further can be a subcellular fraction or extract, or a crude or substantially pure nucleic acid or protein preparation. A sample can be prepared by methods known in the art suitable for the particular format of the detection method employed.

The methods of detecting an NR3B nucleic acid molecule in a sample can be either qualitative or quantitative, and can detect the presence, abundance, integrity or structure of the nucleic acid molecule, as desired for a particular application. Suitable hybridization-based assay methods include, for example, in situ hybridization, which can be used to detect altered chromosomal location of the nucleic acid molecule, altered gene copy number, and RNA abundance, depending on the assay format used. Other hybridization methods include, for example, Northern blots and RNase protection assays, which can be used to determine the abundance and integrity of different RNA splice variants, and Southern blots, which can be used to determine the copy number and integrity of DNA. A hybridization probe can be labeled with any suitable detectable moiety, such as a radioisotope, fluorochrome, chemiluminescent marker, biotin, or other detectable moiety known in the art that is detectable by analytical methods.

Suitable amplification-based detection methods are also well known in the art, and include, for example, qualitative or quantitative polymerase chain reaction (PCR); reverse-transcription PCR (RT-PCR); single strand conformational polymorphism (SSCP) analysis, which can readily identify a single point mutation in DNA based on differences in the secondary structure of single-strand DNA that produce an altered electrophoretic mobility upon non-denaturing gel electrophoresis; and coupled PCR, transcription and translation assays, such as a protein truncation test, in which a mutation in DNA is determined by an altered protein product on an electrophoresis gel. The amplified nucleic acid molecule can be sequenced to detect mutations and mutational hot-spots, and specific PCR-based assays for large-scale screening of samples to identify such mutations can be developed.

The invention also provides isolated NR3B polypeptides and functional fragments therefrom, having amino acid sequences as described above with respect to polypeptides encoded by invention nucleic acid molecules. NR3B polypeptides and functional fragments can be used, for example, in therapeutic applications in which such polypeptides and fragments are administered onto or into cells; in screening assays to identify ligands, agonists and antagonists of excitatory glycine receptors; in research applications to identify additional NR3B-associating polypeptides; to raise antibodies for use in diagnostic and prognostic methods; to affinity purify antibodies and ligands; and in other applications known to those skilled in the art.

An isolated NR3B polypeptide of the invention can optionally contain amino acids with various chemical or enzymatic modifications with respect to naturally occurring amino acids. Such modifications can enhance the stability, bioactivity, immunogenicity or other advantageous property of an invention polypeptide. Thus, a polypeptide can contain an amino acid modified by replacement of hydrogen by an alkyl, acyl, or amino group; by esterification of a carboxyl group with a suitable alkyl or aryl moiety; by alkylation of a hydroxyl group to form an ether derivative; by phosphorylation or dephosphorylation of a serine, threonine or tyrosine residue; by N- or O-linked glycosylation; by iodination; by radiolabeling; or the like. A polypeptide can also include a modified amino acids such as hydroxyproline or carboxyglutamate, or a D-amino acid in place of its corresponding L-amino acid. Those skilled in the art can determine an appropriate amino acid modification for a given application.

In another embodiment, the invention provides an isolated NR3B peptide. An exemplary NR3B peptide contains at least 8 contiguous amino acids of a naturally occurring NR3B polypeptide, such as at least 8 contiguous amino acids of SEQ ID NOS:2, 4, 6 or 8. Such a peptide can contain, for example, at least about 10, 12, 15, 20, 25, 30, 35, 40, 50, 60, 70, 80, 90, 100, 110, 125, 150, 200, 250, 300, 400, 500, 600, 700, 800, 900, 950 or more amino acids, up to the full length of the reference polypeptide. A peptide of at least about 8 amino acids can be used, for example, as an immunogen to raise antibodies specific for an NR3B polypeptide, or as an antigen to purify antibodies specific for an NR3B polypeptide. When used as an antigen, an invention peptide can be attached to a carrier molecule such as bovine serum albumin (BSA) or keyhole limpet hemocyanin (KLH).

Peptides that are likely to be antigenic or immunogenic can be predicted using methods and algorithms known in the art and described, for example, by Irnaten et al., Protein Eng. 11:949-955 (1998), and Savoie et al., Pac. Symp. Biocomput. 1999:182-189 (1999). Immunogenicity of the peptides of the invention can be determined by methods known in the art, such as assay of a delayed-type hypersensitivity response in an animal sensitized to a NR3B polypeptide, or by elicitation of antibodies specific for NR3B polypeptides. Likewise, antigenicity of the peptides of the invention can be determined by methods known in the art, such as by ELISA analysis, as described, for example, in Harlow and Lane, Antibodies: A Laboratory Manual, Cold Spring Harbor Laboratory Press (1988).

The isolated NR3B polypeptides, functional fragments and peptides of the invention can be prepared by methods known in the art, including biochemical, recombinant and synthetic methods. For example, polypeptides can be purified by routine biochemical methods from cells or tissues that express the polypeptide. The detection methods disclosed herein can be adapted for determining which cells or tissues are appropriate starting materials. Biochemical purification can include, for example, steps such as solubilization of the appropriate cells, size or affinity chromatography, electrophoresis, and immunoaffinity procedures. The methods and conditions for biochemical purification of a polypeptide of the invention can be chosen by those skilled in the art, and purification monitored, for example, by an ELISA assay or a functional assay.

An NR3B polypeptide, functional fragment or peptide having any desired boundaries can also be produced by recombinant methods. Recombinant methods involve expressing a nucleic acid molecule encoding the desired polypeptide or fragment in a host cell or cell extract, and isolating the recombinant polypeptide or fragment, such as by routine biochemical purification methods described above. To facilitate identification and purification of the recombinant polypeptide, it is often desirable to insert or add, in-frame with the coding sequence, nucleic acid sequences that encode epitope tags, polyhistidine tags, glutathione-S-transferase (GST) domains, fluorescent proteins (e.g. EGFP) and the like. Methods for producing and expressing recombinant polypeptides in vitro and in prokaryotic and eukaryotic host cells are well known in the art.

Thus, the invention provides a method of producing an NR3B polypeptide or functional fragment either in vitro or in a cell, by expressing a nucleic acid molecule encoding the polypeptide or fragment under appropriate conditions. Optionally, the polypeptide or fragment so produced can be partially purified, such as obtained as a membrane extract.

The invention NR3B polypeptide fragments and peptides can also be produced, for example, by enzymatic or chemical cleavage of the full-length polypeptide. Methods for enzymatic and chemical cleavage and for purification of the resultant peptide fragments are well known in the art (see, for example, Deutscher, Methods in Enzymology, Vol. 182, “Guide to Protein Purification,” San Diego: Academic Press, Inc. (1990)).

Depending on the intended application, the isolated NR3B polypeptide or functional fragment can optionally be isolated in, or reconstituted into, a natural or artificial lipid bilayer, such as a cell membrane or liposome, with or without other cellular components. Thus, in one embodiment, the invention provides an isolated NR3B polypeptide, further comprising a membrane, and optionally further comprising an NMDA receptor subunit, such as an NR1 subunit. Membrane-associated NR3B polypeptides and functional fragments are useful in applications in which structural integrity of the subunit and/or excitatory glycine receptor are important, such as in the screening assays described herein.

The invention also provides an antibody or antigen binding fragment thereof which specifically binds an NR3B polypeptide. Such antibodies, which include polyclonal, monoclonal, chimeric, bifunctional, and humanized antibodies, can be used, for example, to affinity purify an NR3B polypeptide or functional fragment; to detect cellular polypeptides, including NMDA receptor subunits, that associate with NR3B; and in therapeutic, diagnostic and research applications known to those skilled in the art and described further below. An antibody that “specifically binds” and NR3B polypeptide binds with high affinity to a NR3B polypeptide in a binding assay, such as an immunoblot or ELISA assay, without substantial cross-reactivity with other polypeptides such as other NMDA receptor subunits.

An “antigen binding fragment” of an antibody of the invention includes, for example, individual heavy or light chains and fragments thereof, such as VL, VH and Fd; monovalent fragments, such as Fv, Fab, and Fab′; bivalent fragments such as F(ab′)₂; single chain Fv (scFv); and Fc fragments. Antigen binding fragments include, for example, fragments produced by protease digestion or reduction of an antibody, as well as fragments produced by recombinant DNA methods known to those skilled in the art.

In one embodiment, the invention provides antibodies and antigen binding fragments thereof that specifically bind an NR3B polypeptide containing an amino acid sequence designated SEQ ID NO:2, 4, 6 or 8. For certain applications, such as to antagonize receptor activity, antibodies that bind an extracellular portion of NR3B are desirable, including antibodies that bind the region N-terminal to the S1 domain, the S1 domain, the S2 domain, or the region between the M3 and M4 domains. For other applications, antibodies that distinguish a splice variant form of an NR3B receptor are useful, such as antibodies that bind the 15 amino acid portion of rat NR3B B4 not present in the NR3B A2 form.

The antibodies of the invention can be produced by any suitable method known in the art. For example, a NR3B polypeptide or immunogenic peptide of the invention, or a nucleic acid expressing such a polypeptide or peptide, can be administered to an animal, using standard methods, and polyclonal antibodies isolated therefrom. Such polypeptides or peptides, if desired, can be conjugated to a carrier, such as KLH, serum albumin, tetanus toxoid and the like, using standard linking techniques, to increase their immunogenicity. Additionally, such peptides can be formulated together with an adjuvant known in the art, such as Freund's complete or incomplete adjuvant. The antibodies so generated can be used in the form of serum isolated from an immunized animal, or the antibody can be affinity purified from the serum using the invention peptides or polypeptides.

Additionally, the antibodies of the invention can be monoclonal antibodies produced by a hybridoma cell line, by chemical synthesis, or by recombinant methods. Modified antibodies, such as chimeric antibodies, single chain antibodies, humanized antibodies and CDR-grafted or bifunctional antibodies, can also be produced by methods well known to those skilled in the art.

Methods of preparing and using antibodies and antigen-binding fragments, including detectably labeled antibodies, are described, for example, in Harlow and Lane, Antibodies: A Laboratory Manual, Cold Spring Harbor Laboratory, New York (1989); in Day, E. D., Advanced Immunochemistry, Second Ed., Wiley-Liss, Inc., New York, N.Y. (1990); and in Borrebaeck (Ed.), Antibody Engineering, Second Ed., Oxford University Press, New York (1995). Example V describes a method for producing NR3B-specific antibodies.

The invention also provides a method for detecting an NR3B polypeptide in a sample. The method is practiced by contacting a sample with an antibody specific for an NR3B polypeptide and detecting specific binding of the antibody to the sample. As described above with respect to encoding nucleic acid molecules, altered expression, abundance or integrity of an NR3B polypeptide in a sample can thus be indicative of a pathological condition, including any of the acute or chronic neurological and neurodegenerative conditions described herein.

The methods of detecting an NR3B polypeptide in a sample can be either qualitative or quantitative, and can detect the presence, abundance, integrity or localization of the polypeptide, as desired for a particular application. Contemplated assays to detect a polypeptide in a sample include in situ histochemistry, immunoblotting, immunoprecipitation, FACS analysis, radioligand binding, and ELISA analysis. Such assays can be direct, using a detectably labeled ligand, or indirect, using a labeled secondary reagent, such as an anti-ligand antibody. Exemplary labels include fluorescent labels, enzymes, radioisotopes, and biotin. Detection can be by any convenient analytical means, including by spectrophotometric, radiographic or chemiluminescent means, depending on the assay.

The invention also provides a method of detecting an NR3B ligand. In one embodiment, the method is practiced by contacting an NR3B polypeptide with one or more compounds under conditions suitable for detecting binding to the polypeptide, and detecting a candidate compound that binds the polypeptide. In another embodiment, the invention is practiced by contacting an NR3B functional fragment with one or more compounds under conditions suitable for detecting binding to the fragment, and detecting a candidate compound that binds the fragment. For use in such an application, the NR3B functional fragment desirably contains at least one extracellular domain of the NR3B polypeptide, such as the region N-terminal to the S1 domain, the S1 domain, the S2 domain, or the region between the M3 and M4 domains (see FIG. 8).

The invention further provides a method of detecting an excitatory glycine receptor ligand. The method is practiced by contacting an excitatory glycine receptor with one or more compounds under conditions suitable for detecting binding to the receptor, and detecting a candidate compound that binds the receptor.

In one embodiment, the excitatory glycine receptor contains an NR3B polypeptide and an NR1 polypeptide. In another embodiment, the excitatory glycine receptor contains an NR3A polypeptide and an NR1 polypeptide. In a further embodiment, the excitatory glycine receptor contains an NR3B polypeptide, an NR3A polypeptide and an NR1 polypeptide. In yet another embodiment, the excitatory glycine receptor contains an NR3B and/or an NR3A polypeptide, an NR1 polypeptide and an NR2 polypeptide (i.e. an NR2A, 2B, 2C or 2D polypeptide).

As disclosed herein, co-expression of an NR3A and an NR1 polypeptide also results in the formation of excitatory glycine receptors. Rat NR3A polypeptides and encoding nucleic acid molecules are known in the art, and exemplary molecules have the nucleotide and predicted amino acid sequences set forth in Table 2:

TABLE 2

| SUBUNIT | ACCESSION NUMBER |
|---|---|
| Rat NR3A (NMDAR-L) | U29873 |
| Rat NR3A (x-1) | L34938 |
| Rat NR3A (splicing variant) | AF061945 |
| Rat NR3A (splicing variant) | AF073379 |

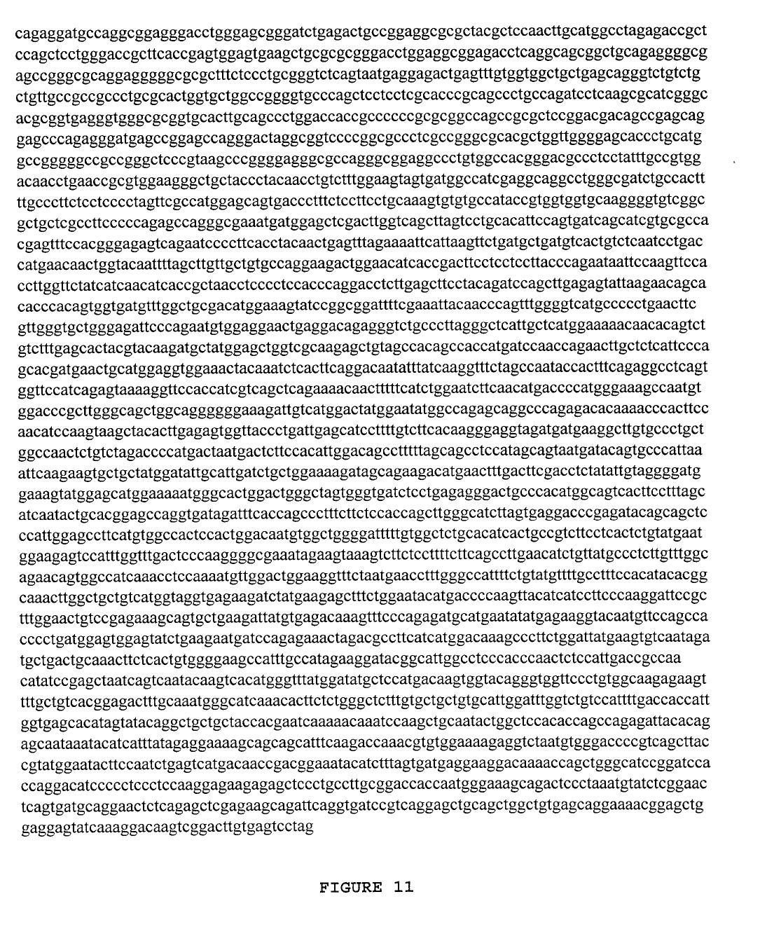

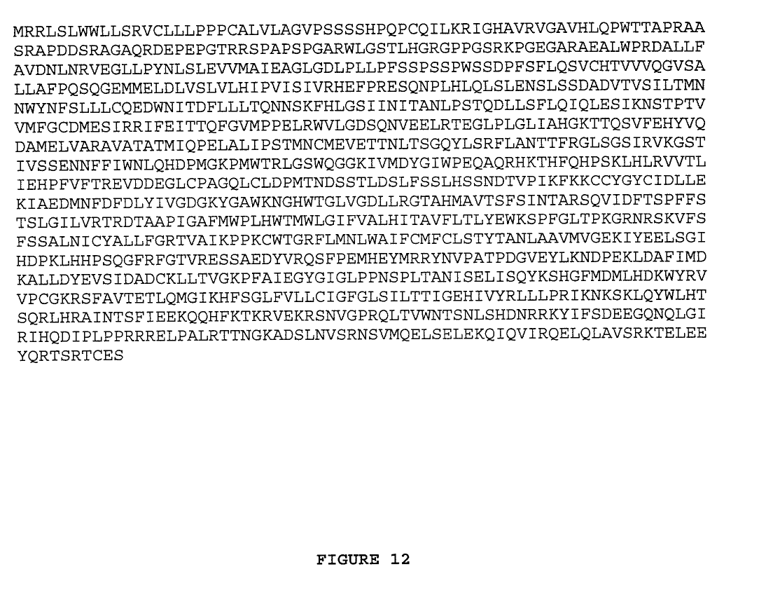

Disclosed herein as SEQ ID NO:53 is the predicted human NR3A cDNA sequence and its encoding amino acid sequence (SEQ ID NO:54) (see FIGS. 11 and 12). These sequence were predicted from the human NR3A genomic sequences in the database (GenBank Accession Nos. NT_{—}025809, AL35616, XM042803.1 and AL359651.1) based on the rat NR3A cDNA and amino acid sequences. The skilled person can likewise determine additional NR3A sequences from other species.

NR2 polypeptides and encoding nucleic acid molecules are also known in the art, and exemplary molecules have the nucleotide and predicted amino acid sequences set forth in Table 3:

TABLE 3

| SUBUNIT | ACCESSION NUMBER |
|---|---|
| Human NR2A cDNA | NM_000833, U09002, U90277, |
| | XM_030214, XM_030215, |
| | XM_007911, XM_016282 |
| Human NR2A genomic | AC007218, AC006531 |
| Rat NR2A cDNA | NM_012573, M91561, AF001423, |
| | D13211 |
| Mouse NR2A (epsilon 1) | NM_008170, D10217 |
| Human NR2B cDNA | XM_006636, XM_042404, |
| | XM_042405, XM_042406, |
| | NM_000834, U88963, U11287, |
| | U90278 |
| Human NR2B genomic | AC007535 |
| Rat NR2B cDNA | NM_012574, U11419, M91562 |
| Mouse NR2B (epsilon 2) | NM_008171, D10651 |
| Human NR2C cDNA | NM_000835, U77782, XM_046949, |
| | L76224, XM_012596 |
| Rat NR2C | NM_012575, D13212, M91563, |
| | U08259 |
| Mouse NR2C (epsilon 3) | NM_010350, D10694 |
| Human NR2D cDNA | XM_009108, NM_000836, U77783 |
| Human NR2D genomic | AC011527, AC008403 |
| Rat NR2D | U08260, NM_022797, L31612, |
| | L31611, D13213 |
| Mouse NR2D (epsilon 4) | NM_008172, D12822 |

The skilled person will appreciate that for use in the methods described herein, an NR3A or NR2 polypeptide can have modifications to a naturally occurring sequence, or be a functional fragment of the naturally occurring sequence, so long as the desired biological activity is retained. An exemplary modification of a naturally occurring NR3A or NR2 polypeptide that does not affect biological activity is the addition of an epitope tag to facilitate identification in procedures such as immunolocalization and immunoprecipitation.

NR3A biological activities include, for example, the ability to oligomerize with other NMDA subunits, including NR1; the ability to bind glycine; and the ability to form excitatory glycine receptors in association with NR1. NR2 biological activities include, for example, the ability to oligomerize with other NMDA subunits, including NR1; the ability to bind glutamate; and the ability to form excitatory glutamate receptors in association with NR1.

As described above with respect to NR3B and NR1 polypeptides and functional fragments, the skilled person can readily make NR3A and NR2 molecules with sequences that differ from the naturally occurring sequence and test such molecules to confirm that a desired NR3A or NR2 biological activity is retained.

As used herein, the term “ligand” refers to any biological or chemical compound that binds the recited polypeptide, fragment or receptor with high affinity. High affinity binding refers to binding with a Kd of less than about 10⁻³M, such as less than 10⁻⁵M, and often less than 10⁻⁷M. Glycine and NR3B antibodies are examples of ligands of NR3B.

An “NR3B ligand” or “excitatory glycine receptor ligand” can further be an agonist or antagonist of an excitatory glycine receptor, as described below, or can be a compound having little or no effect on excitatory glycine receptor biological activity. For example, a ligand without agonistic or antagonistic activity can be used to specifically target a diagnostic or therapeutic moiety to cells and tissues that express an excitatory glycine receptor. Thus, an identified ligand can be labeled with a detectable moiety, such as a radiolabel, fluorochrome, ferromagnetic substance, or luminescent substance, and used to detect normal or abnormal expression of an excitatory glycine receptor in an isolated sample or in in vivo diagnostic imaging procedures. Likewise, an identified ligand can be labeled with a therapeutic moiety, such as a cytotoxic or cytostatic agent or radioisotope, and administered in an effective amount to arrest proliferation or kill a cell or tissue that aberrantly expresses an excitatory glycine receptor for use in therapeutic applications described further below.

Binding assays, including high-throughput automated binding assays, are well known in the art and can be used in the invention methods. The assay format can employ a cell, cell membrane, artificial membrane system, or purified polypeptide, fragment or receptor, either in solution or attached to a solid phase. If desired, the binding assay can be performed in the presence of a known ligand of NR3B or of an excitatory glycine receptor, such as glycine.

Suitable assays that can be used for detecting ligand binding include, for example, scintillation proximity assays (SPA) (Alouani, Methods Mol. Biol. 138:135-41 (2000)), UV or chemical cross-linking (Fancy, Curr. Opin. Chem. Biol. 4:28-33 (2000)), competition binding assays (Yamamura et al., Methods in Neurotransmitter Receptor Analysis, Raven Press, New York, 1990), biomolecular interaction analysis (BIA) (Weinberger et al., Pharmacogenomics 1:395-416 (2000)), mass spectrometry (MS) (McLafferty et al., Science 284:1289-1290 (1999) and Degterev, et al., Nature Cell Biology 3:173-182 (2001)), nuclear magnetic resonance (NMR) (Shuker et al., Science 274:1531-1534 (1996), Hajduk et al., J. Med. Chem. 42:2315-2317 (1999), and Chen and Shapiro, Anal. Chem. 71:669A-675A (1999)), fluorescence polarization assays (FPA) (Degterev et al., supra, 2001); surface plasmon resonance (SPR)(Liparoto et al., J. Mol Recognit. 12:316-321 (1999)); and protein chip proteomic array analysis (e.g. ProteinChip™ System from Ciphergen Biosystems, which can be used in tandem with mass spectrometry analysis for sequence or structure determination).

An exemplary assay that has been used successfully to identify ligands of an NMDA receptor is phage display (see Li et al., Nature Biotech. 14:986-991 (1996), which describes contacting an N-terminal fragment of an NR1 polypeptide with a phage display library). A similar phage display approach can be applied to determine NR3B ligands and excitatory glycine receptor ligands.

Exemplary high-throughput receptor binding assays are described, for example, in Mellentin-Micelotti et al., Anal. Biochem. 272:P182-190 (1999); Zuck et al., Proc. Natl. Acad. Sci. USA 96:11122-11127 (1999); and Zhang et al., Anal. Biochem. 268;134-142 (1999). Other suitable methods are known in the art.

The invention also provides methods of detecting an excitatory glycine receptor agonist or antagonist The method is practiced by contacting an excitatory glycine receptor under conditions suitable for detecting excitatory glycine receptor activation, and detecting a candidate compound that alters excitatory glycine receptor activation. As described herein, excitatory glycine receptor activation can be evidenced by elicitation of a monovalent cation current, with little or no channel permeability to Ca²⁺ and little or no inhibition by Mg²⁺.

In one embodiment, the excitatory glycine receptor contains an NR3B polypeptide and an NR1 polypeptide. In another embodiment, the excitatory glycine receptor contains an NR3A polypeptide and an NR1 polypeptide. In a further embodiment, the excitatory glycine receptor contains an NR3B polypeptide, an NR3A polypeptide and an NR1 polypeptide. In yet another embodiment, the excitatory glycine receptor contains an NR3B polypeptide, an NR1 polypeptide and an NR2 polypeptide (i.e. an NR2A, 2B, 2C or 2D polypeptide). In another embodiment, the excitatory glycine receptor contains an NR3A polypeptide, an NR1 polypeptide and an NR2 polypeptide (i.e. an NR2A, 2B, 2C or 2D polypeptide).

The agonists and antagonists identified by the methods of the invention are useful in therapeutic applications, described further below, in which it is desirable to increase or decrease ion flow through the excitatory glycine receptor.

As used herein, the term “excitatory glycine receptor agonist” refers to a compound that increases or activates excitatory glycine receptor cation currents. An agonist can act by any mechanism, such as by binding the receptor at the normal glycine binding site, thereby mimicking glycine and promoting receptor activation. An agonist can also act, for example, by potentiating the activity of glycine, or by favorably altering the conformation of the receptor. The methods of the invention can be used to detect agonists that act through any agonistic mechanism.

As used herein, the term “excitatory glycine receptor antagonist” refers to a compound that decreases or inhibits excitatory glycine receptor cation currents. Typically, the effect of an antagonist is observed as a blocking of activation by an agonist. For example, 5, 7-di-Cl-Kynurenate blocks glycine activated currents through the NR3B/NR1 receptor.

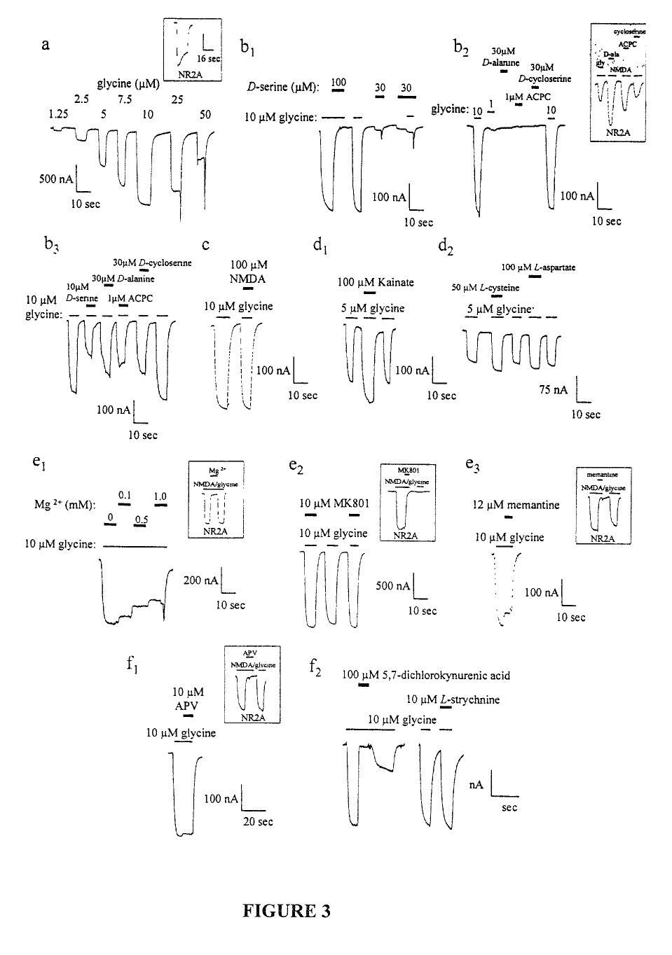

Antagonists include, for example, partial antagonists, partial agonists, competitive antagonists, non-competitive antagonists and uncompetitive antagonists. A competitive antagonist interacts with or near the site specific for the agonist. A non-competitive antagonist inactivates the function of the receptor by interacting with a site other than the site that interacts with the agonist. Partial agonists have both agonistic and antagonistic activity. For example, as shown in FIG. 3, D-serine evokes small NR1/NR3B currents alone, but dose-dependently inhibits currents in the presence of glycine. The methods of the invention can be used to detect antagonists that act through any antagonistic mechanism.

Methods of detecting excitatory glycine receptor agonists and antagonists can advantageously be performed either in the presence or absence of a physiologically relevant excitatory glycine receptor agonist such as glycine. Compounds that demonstrate agonistic and antagonistic effects in the presence of glycine are particularly useful for use in therapeutic applications, in which physiological concentrations of circulatory glycine are present. In such methods, concentrations of glycine of about 1 to about 100 μM, such as about 5-10 μM, are suitable.

Electrophysiological methods for detecting monovalent cation currents through an excitatory glycine receptor are well known in the art. Exemplary methods for recording whole-cell and single-channel currents in Xenopus oocytes, brain slices, mammalian cells and cell-free membrane patches are described in Das et al., Nature 393:377-381 (1998); Sakmann and Neherand, in Single-Channel Recording, 2nd ed., Ch. 15, pp. 341-355, (1995), edited by Bert Sakmann and Erwin Neher, Plenum Press, New York; Penner, in Single-Channel Recording, 2nd ed., Ch. 1, pp. 3-28; Hamill et al., Pflugers Arch. 391:85-100 (1981); Ilers et al., in Single-Channel Recording, 2nd ed., Ch. 9, pp. 213-229, (1995), edited by Bert Sakmann and Erwin Neher, Plenum Press, New York; and in the Examples, below.

Ionic currents can also be detected using suitable detectably labeled ion indicators. Ion indicators and methods for their use are known in the art. For example, monovalent cation currents through the excitatory glycine receptor can be detected using Na⁺ or K⁺ ion indicators, which can be fluorescently labeled or radiolabeled (see, for example, Moore et al., Proc. Natl. Acad. Sci. USA 90:8058-8062 (1993); Paucek et al., J. Biol. Chem. 267:26062-26069 (1992); Xu et al., J. Biol. Chem. 270: 19606-19612 (1995)). Exemplary ion indicators include: SBFI sodium indicator, Sodium Green sodium indicator; CoroNa Red sodium indicator; PBFI potassium indicator; 6-Methoxy-N-(3-sulfopropyl)quinolinium (SPQ) chloride indicator; N-(Ethoxycarbonylmethyl)-6-methoxyquinolinium bromide (MQAE) chloride indicator; 6-Methoxy-N-ethylquinolinium iodide (MEQ) chloride indicator; Lucigenin chloride indicator, which are available from Molecular Probes, Inc.

Subsequent to excitatory glycine receptor activation and membrane depolarization, an influx of Ca²⁺ ions occurs if voltage-dependent Ca²⁺ channels are present in the cell being studied. If the cell of interest does not endogenously express voltage-dependent Ca²⁺ channels, the cell can be recombinantly engineered to express such channels, using voltage-dependent Ca²⁺ channel subunit gene sequences and molecular biology methods known in the art. Accordingly, ionic currents through the excitatory glycine receptor can also be detected, indirectly, using detectably labeled Ca²⁺ ion indicators, which can be fluorescently labeled or radiolabeled. Exemplary Ca²⁺ ion indicators include FLUO-3 AM, FLUO-4 AM, FURA-2, INDO-1, FURA RED, CALCIUM GREEN, CALCIUM ORANGE, CALCIUM CRIMSON, BTC, and OREGON GREEN BAPTA (see, for example, Grynkiewitz et al., J. Biol. Chem. 260:3440-3450 (1985); Sullivan et al., in Calcium Signal Protocol, Methods in Molecular Biology 114: 125-133, Edited by David G. Lambert, Human Press, Totowa, N.J. (1999); Miyawaki et al., Proc. Natl. Acad. Sci. USA 96:2135-2140 (1999); and Coward et al., Analyt. Biochem. 270:242-248 (1999)).

Assay methods for identifying compounds that bind to or modulate excitatory glycine receptor activity (e.g. ligands, agonists and antagonists) generally involve comparison to a control. One type of a “control” is a NR3B polypeptide or excitatory glycine receptor that is treated substantially the same as the polypeptide or receptor exposed to the candidate compound, except the control is not exposed to the candidate compound. For example, the same recombinant cell can be tested in the presence and absence of candidate compound, by merely changing the solution contacting the cell. Another type of “control” is a cell that is essentially identical to the NR3B polypeptide- or excitatory glycine receptor-expressing recombinant cell, except the control cell does not express the polypeptide or receptor. In this situation, the response of the test cell to a candidate compound is compared to the response (or lack of response) of the control cell to the same compound under substantially the same reaction conditions.

The term “candidate compound” refers to any molecule that potentially acts as a ligand, agonist or antagonist or ligand in the screening methods disclosed herein. A candidate compound can be a naturally occurring macromolecule, such as a polypeptide, amino acid, nucleic acid, carbohydrate, lipid, or any combination thereof. A candidate compound also can be a partially or completely synthetic derivative, analog or mimetic of such a macromolecule, or a small organic molecule prepared by combinatorial chemistry methods. If desired in a particular assay format, a candidate compound can be detectably labeled or attached to a solid support.

Methods for preparing large libraries of compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

The number of different candidate compounds to test in the methods of the invention will depend on the application of the method. For example, one or a small number of candidate compounds can be advantageous in manual screening procedures, or when it is desired to compare efficacy among several predicted ligands, agonists or antagonists. However, it is generally understood that the larger the number of candidate compounds, the greater the likelihood of identifying a compound having the desired activity in a screening assay. Additionally, large numbers of compounds can be processed in high-throughput automated screening assays. Therefore, “one or more candidate compounds” can be, for example, 2 or more, such as 5, 10, 15, 20, 50 or 100 or more different compounds, such as greater than about 10³, 10⁵ or 10⁷ different compounds, which can be assayed simultaneously or sequentially.

The function of the NR3A polypeptide has remained elusive, despite years of investigation (see Ciaberra et al., J. Neuroscience 15:6498-6508 (1995); Sucher et al., J. Neuroscience 15:6509-6520 (1995); Das et al. supra (1998); Perez-Otano et al., J. Neuroscience 21:1228-1237 (2001)). As disclosed herein, when expressed in combination with NR1 and optionally also NR2 subunits, the NR3A polypeptide, like the NR3B polypeptide, forms an excitatory glycine receptor (see Example IV). The invention thus provides isolated excitatory glycine receptors containing either an NR3A polypeptide or an NR3B polypeptide, or both. Such receptors are suitable use in the methods described herein of detecting excitatory glycine receptors agonists, antagonists and ligands. As described previously, depending on the particular assay, the isolated receptors can optionally be present at the surface of an intact cell, present in a cell membrane with or without other cellular components, or reconstituted into a natural or artificial lipid bilayer.

In one embodiment, the excitatory glycine receptor contains an NR3B polypeptide and an NR1 polypeptide. In another embodiment, the excitatory glycine receptor contains an NR3A polypeptide and an NR1 polypeptide. In a further embodiment, the excitatory glycine receptor contains an NR3B polypeptide, an NR3A polypeptide and an NR1 polypeptide. In yet another embodiment, the excitatory glycine receptor contains an NR3B polypeptide, an NR1 polypeptide and an NR2 polypeptide (i.e. an NR2A, 2B, 2C or 2D polypeptide). In another embodiment, the excitatory glycine receptor contains an NR3A polypeptide, an NR1 polypeptide and an NR2 polypeptide (i.e. an NR2A, 2B, 2C or 2D polypeptide).

Suitable NR3B, NR3A, NR2A-D and NR1 polypeptides that can be used as subunits of excitatory glycine receptors, including both naturally occurring polypeptides, and modifications and functional fragments of such polypeptides, have been described previously.

The invention also provides therapeutic methods for the prevention and amelioration of conditions in which inappropriate NMDA receptor activation, or inappropriate responses to glycine or glutamate, are implicated. Such conditions include, for example, acute neurologic condition, such as cerebral ischemia; stroke; hypoxia; anoxia; poisoning by carbon monoxide, manganese, cyanide or domoic acid; hypoglycemia; mechanical trauma to the nervous system such as trauma to the head or spinal cord; or epileptic seizure. Other conditions include, for example, chronic neurodegenerative disease, such as Huntington's disease; a disorder of photoreceptor degeneration such as retinitis pigmentosa; acquired immunodeficiency syndrome (AIDS) dementia complex (HIV-associated dementia); a neuropathic pain syndrome such as causalgia or a painful peripheral neuropathy; olivopontocerebellar atrophy; Parkinsonism; amyotrophic lateral sclerosis; a mitochondrial abnormality or other biochemical disorder such as MELAS syndrome, MERRF, Leber's disease, Wernicke's encephalopathy, Rett syndrome, homocysteinuria, hyperhomocysteinemia, hyperprolinemia, nonketotic hyperglycinemia, hydroxybutyric aminoaciduria, sulfite oxidase deficiency, combined systems disease, lead encephalopathy, Alzheimer's disease, hepatic encephalopathy, Tourette's syndrome, drug addiction/tolerance/dependency, glaucoma, depression, anxiety, multiple sclerosis and other demyelinating disorders. Other conditions are known in the art and reviewed, for example, in Lipton et al., New Engl. J. Med. 330:613-622 (1994) and Cull-Candy et al., Curr. Opin. Neurobiol. 11:327-335 (2001).

Thus, the invention provides methods for increasing or decreasing signaling through an excitatory glycine receptor by administering an excitatory glycine receptor agonist, antagonist or ligand, or an NR3B ligand, to an individual. Methods of identifying such agonist, antagonist and ligands have been described previously.

The invention also provides gene therapy methods for modulating a cellular response to glycine or glutamate. By overexpressing a full-length NR3B polypeptide, the NR1 subunit can form NR1/NR3B receptors, which are insensitve to glutamate, rather than NR1/NR2 receptors. Therefore, detrimental glutamate responses can be reduced. In contrast, by expressing a dominant negative NR3B polypeptide, or a construct that prevents translation of endogenous NR3B, fewer functional NR1/NR3B receptors will be formed. Therefore, detrimental glycine responses can be reduced. Therapeutic applications in which it is desirable to modulate cellular responses to glutamate and glycine are described above.

In one embodiment, the invention provides a method of modulating a cellular response to glycine or glutamate by introducing a nucleic acid molecule encoding an NR3B polypeptide or functional fragment into a cell, and expressing the NR3B functional fragment encoded by the nucleic acid molecule in the cell. In another embodiment, the invention provides a method of modulating a cellular response to glycine or glutamate by introducing an antisense nucleic acid molecule, a ribozyme molecule or a small interfering RNA (siRNA) molecule into the cell, wherein the molecule hybridizes to an NR3B nucleic acid molecule and prevents translation of the encoded NR3B polypeptide.

Suitable gene therapy vectors have been described previously. For gene therapy applications, the nucleic acid molecule can be administered to a subject by various routes. For example, local administration at the site of a pathology can be advantageous because there is no dilution effect and, therefore, the likelihood that a majority of the targeted cells will be contacted with the nucleic acid molecule is increased. This is particularly true in the eye, where either intravitreal or intraretinal administration is possible. In addition, administration can be systemic, such as via intravenous or subcutaneous injection into the subject. For example, following injection, viral vectors will circulate until they recognize host cells with the appropriate target specificity for infection.

Receptor-mediated DNA delivery approaches also can be used to deliver a nucleic acid molecule into cells in a tissue-specific manner using a tissue-specific ligand or an antibody that is non-covalently complexed with the nucleic acid molecule via a bridging molecule. Direct injection of a naked nucleic acid molecule or a nucleic acid molecule encapsulated, for example, in cationic liposomes also can be used for stable gene transfer into non-dividing or dividing cells. In addition, a nucleic acid molecule can be transferred into a variety of tissues using the particle bombardment method.

Antisense nucleotide sequences that are complementary to a nucleic acid molecule encoding an NR3B polypeptide can be used to prevent or reduce NR3B expression. Therefore, the method can be practiced with an antisense nucleic acid molecule complementary to at least a portion of the nucleotide sequence of SEQ ID NOS:1, 3, 5 or 7. For example, the antisense nucleic acid molecule can be complementary to a region within the N-terminus of SEQ ID NOS:1, 3, 5 or 7, such as within nucleotides 1-1000, 1-500, 1-100 or 1-18, and can optionally include sequences 5′ to the start codon. Methods of preparing antisense nucleic acids molecules and using them therapeutically are known in the art and described, for example, in Galderisi et al., J. Cell Physiol. 181:251-257 (1999).

Likewise, ribozymes that bind to and cleave SEQ ID NOS:1, 3, 5 or 7 can also be effective in preventing or reducing NR3B expression. Methods of preparing ribozymes and DNA encoding ribozymes, including hairpin and hammerhead ribozymes, and using them therapeutically are known in the art and described, for example, in Lewin et al., Trends Mol. Med. 7:221-228 (2001).

Additionally, small interfering RNAs (siRNAs), which are short duplex RNAs with overhanging 3′ ends, directed against SEQ ID NOS:1, 3, 5 or 7, can also be effective in preventing or reducing NR3B expression. Methods of preparing and using siRNAs are known in the art and described, for example, in Elbashir et al., Nature 411:494-498 (2001).

The therapeutic compounds of the invention, including agonists, antagonists, ligands and nucleic acid molecules, can be formulated and administered in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for a particular therapeutic application in humans can be extrapolated based on the activity of the compound in the in vitro binding and signaling assays described herein, or from recognized animal models of the particular disorder.

The total amount of therapeutic compound can be administered as a single dose or by infusion over a relatively short period of time, or can be administered in multiple doses administered over a more prolonged period of time. Additionally, the compound can be administered in a slow-release matrice, which can be implanted for systemic delivery at or near the site of the target tissue. Contemplated matrices useful for controlled release of therapeutic compounds are well known in the art, and include materials such as DepoFoam™, biopolymers, micropumps, and the like.

The therapeutic compounds can be administered to an individual by routes known in the art including, for example, intravenously, intramuscularly, subcutaneously, intraorbitally, intracapsularly, intraperitoneally, intracisternally, intra-articularly, intracerebrally, orally, intravaginally, rectally, topically, intranasally, transdermally or intravitreally.

Preferably, the therapeutic compounds are administered to a subject as a pharmaceutical composition comprising the compound and a pharmaceutically acceptable carrier. The choice of pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters. A pharmaceutically acceptable carrier can further contain physiologically acceptable compounds that stabilize the compound, increase its solubility, or increase its absorption. Such physiologically acceptable compounds include carbohydrates such as glucose, sucrose or dextrans; antioxidants, such as ascorbic acid or glutathione; chelating agents; and low molecular weight proteins.

For applications that require the compounds and compositions to cross the blood-brain barrier, or to cross the cell membrane, formulations that increase the lipophilicity of the compound are particularly desirable. For example, the compounds of the invention can be incorporated into liposomes (Gregoriadis, Liposome Technology, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer. Additionally, the therapeutic compound can be conjugated to a peptide that facilitates cell entry, such as penetratin (also known as Antennapedia peptide), other homeodomain sequences, or the HIV protein Tat.

It is contemplated that administration of the therapeutic compounds and compositions of the invention will result in some beneficial effect to the individual, such as improved overall neurological function or a specific neurological function; an improvement in the quality of life; a reduction in the severity of the symptoms of the disease; a reduction in the number of diseased cells; prolonged survival, and the like. Indicators of beneficial effect are well known in the art, and an appropriate indicator for a particular application can be determined by the skilled person.

The following examples are intended to illustrate but not limit the present invention.

### EXAMPLE I

This example shows cloning and sequence analysis of NR3B.

Degenerate primers were designed based on the sequences of NR3A and other NMDAR family members. Using degenerate PCR in concert with homology screening of a rat brain cDNA library, two novel cDNA fragments (called 5-2 clone 1 and 5-2 clone 2) were obtained which exhibited significant sequence identity with NR3A, but clearly corresponded to a distinct and previously unidentified gene. The novel gene was therefore designated NR3B.

Based on the sequences of 5-2 clone 1 and 5-2 clone 2, two pairs of more specific, nested primers (f4, TGCTGCTATGGCTACTGCATC (SEQ ID NO:17); r4, ATGACAGCAGCCAGGTTGGCCGT (SEQ ID NO:18); f5, CACACATGGCTGTGACCAGC (SEQ ID NO:19); and r5, AGAATGGCATAGCACAGGTTG (SEQ ID NO:20)) were designed and used to PCR screen a Rapid-Screen rat brain cDNA library panel (OriGene Technologies, Inc., Rockville, Md.). Four independent partial NR3B cDNA clones were thus obtained. The coding regions of three of the clones were identical (one of these was designated clone B4), but the fourth clone (clone A2) had a 45-nucleotide deletion in the 5′ coding region, which may, therefore, represent a splice variant of NR3B. A comparison of the sequences of clones B4, A2, 5-2 clone 1, and 5-2 clone 2 revealed that all overlap, e.g., 5-2 clone 2 spans 49% of the 5′ end, and clones B4 and A2 span 92% of the 3′ end of the full-length NR3B cDNA. Full-length NR3B cDNAs were assembled by ligating the EcoRI-XhoI fragment of 5-2 clone 2 (345 bp) to the XhoI-XbaI fragment of either clone B4 (2847 bp) or clone A2 (2802 bp).

The NR3B (B4 form) cDNA has an open reading frame encoding a peptide of 1003 amino acid (aa) residues. Multiple alignments of the predicted amino acid sequence of NR3B with other glutamate receptor subunits indicated that NR3B is closely related to NR3A (47% identity), but shares less identity with the NR1 and NR2 subfamilies (17 to 21%) (FIG. 1). To a lesser degree, NR3B also shares sequence identity (13 to 16%) with other GluR subunits representing non-NMDAR subunits.

The major structural features of NR3A are highly conserved in NR3B, including the S1 and S2 agonist binding domains, and the membrane spanning regions (FIG. 1). A detailed comparison of the critical residues involved in ligand binding to the NR1 and NR2 subunits revealed that NR3B could potentially share ligand specificity with either or both of these classes of NMDA receptor subunits. Five of the seven amino acid residues required for glycine binding to NR1 and six of the ten amino acid residues required for glutamate binding to NR2A are conserved in NR3B. Furthermore, the M2 region of NR3B is similar to that of NR3A, but strikingly different from those of the NR1 and NR2 subunits. The M2 region forms the re-entrant P-loop which lines the ion channel pore of fully assembled NMDA receptors. These molecular features suggest that the NR3B subunit may confer novel ligand binding and channel gating properties to the NMDA receptor complexes into which it is assembled.

The first 500 aa residues in the extracellular N-terminus and intracellular C-terminus are the most divergent regions between NR3B and other NMDA receptor subunits, including the NR3A subunit. These regions are important for intra-subunit assembly of glutamate receptors and for interaction of the receptor complex with associated proteins. Thus, these differences may uniquely specify the selection of partner subunits, subcellular localization and CNS distribution of the NR3B subunit.

Based on the rat NR3B sequence, the corresponding mouse and human NR3B sequences were identified from an analysis of homologous genomic sequences in the database (Accession numbers AC087114 and AC004528, respectively).

### EXAMPLE II

This example shows analysis of the expression and localization of the NR3B subunit.

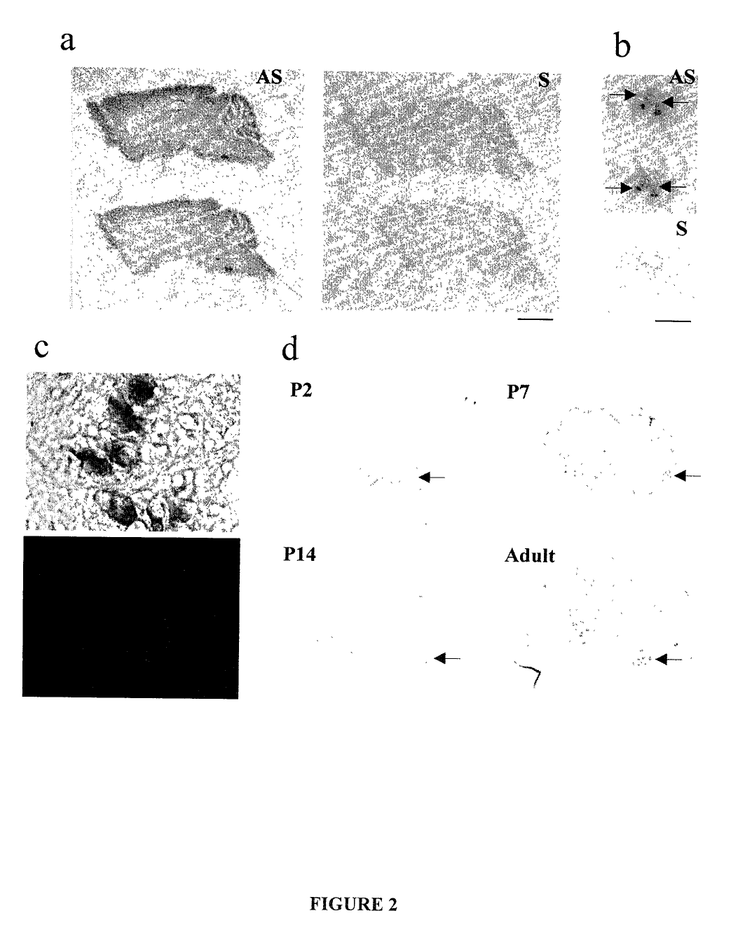

Initially, in situ hybridization was used to detect NR3B mRNA in adult rat brain. Three probes of approximately 400 bp were generated from different N-terminal and C-terminal regions of NR3B. These regions shared little sequence identity with other NMDAR subunits, including NR3A. All three probes detected NR3B signals in the initial experiment. The probe in the N-terminal region giving the strongest signal was chosen for subsequent experiments. ³³P-labeled antisense probes detected “hot spots” in the facial and trigeminal nuclei of the brainstem, and in the ventral horn of the spinal cord (FIGS. 2a, b). Subsequent use of non-isotopic (digoxigenin) labeled probes yielded signals of much higher resolution at the cellular level. NR3B was not detectable in most brain areas, including the cerebrocortex, thalamus, basal ganglia, and hippocampus. In contrast, the spinal cord and the brainstem displayed very strong NR3B signals. In the spinal cord, the NR3B signal was found in motor neurons of the anterior horn at all levels in both layers VIII and IX, which innervate proximal and distal muscle groups, respectively (FIGS. 2c, d). However, an NR3B signal was detected only in specific motor nuclei of the brainstem, specifically the facial and trigeminal nuclei. In addition, some large cells in the vestibular nucleus and reticular formation were also positive for NR3B mRNA.

Further analysis using rats at different ages (P2, P7, P14 and adult) confirmed that NR3B mRNA expression begins at an early postnatal stage (FIG. 2d). A weak NR3B signal was detectable at P2 but increased substantially by P7. The level of NR3B mRNA expression increased postnatally, reaching a peak at P14, and remained elevated into adulthood, consistent with a specific role of NR3B in developing and maintaining motor neuron functions. Based upon these data, it was concluded that NR3B mRNA is localized mainly in motor neurons of the spinal cord and in some motor nuclei of the brainstem. Expression of NR3B protein in these regions has been confirmed by immunocytochemistry.

### EXAMPLE III

This example shows characterization of biological activities of NR3B-containing excitatory glycine receptors.

NR3B cRNA for oocyte expression was produced as follows. The full-length NR3B cDNA was constructed in the pCMV6-XL4 vector (OriGene Technologies, Inc., Rockville, Md.), which contains a T7 promoter upstream of the cDNA insert. Initial attempts to generate an NR3B cRNA using T7 RNA polymerase resulted in the formation of both full-length and truncated species. The truncated cRNA could potentially encode a dominant negative form of NR3B. Therefore, a new vector was constructed to facilitate production of exclusively full-length NR3B cRNA. Construction of this vector proceeded as follows. NR1 cDNA was excised from the pGEM-HE/NR1 vector (a gift from S. F. Heinemann) by digestion with EcoRI and the two ends of the truncated pGEM-HE vector were re-ligated. The truncated pGEM-HE vector retained the 5′ and 3′ UTRs of the X. laevis β-globin gene for high level protein expression in frog oocytes. The SfoI-KpnI fragment of truncated pGEM-HE, which contains the T7 promoter, was replaced with the PvuII-KpnI fragment of pBluescript II KS, which contains the T3 promoter, to generate pJC32. The abbreviated multiple cloning site of pJC32 was then replaced with the multiple cloning site from pcDNA3.1(+) (Invitrogen, Carlsbad, Calif.) by ligating the 102 bp PmeI fragment of pcDNA3.1(+) into the 3 kb SmaI-EcoRV fragment of pJC32 to generate pJC34. Oligos encoding restriction sites for AscI, PacI, and PmeI (ctagcGGCGCGCCTTAATTAAGTTTAAACg (SEQ ID NO:52) and ctagcGTTTAAACTTAATTAAGGCGCGCCg (SEQ ID NO:53)) were annealed and ligated into the NheI site of pJC34. The resulting vector, pJC39, contains three rare restriction sites downstream of the multiple cloning site to facilitate linearization prior to in vitro transcription using T3 RNA polymerase.

The full-length NR3B (B4 form) cDNA was then excised from pCMV6-XL4 by digestion with EcoRI and XbaI, and the 3.2 kb fragment was ligated into EcoRI/XbaI-digested pJC39. The resulting vector, pJC42, was linearized with PmeI, and in vitro transcription was performed using the mMessage mMachine T3 kit (Ambion, Inc., Austin, Tex.) according to the manufacturer's instructions. The cRNA product consisted of a single species with a molecular weight of approximately 1.5 kb.

Microinjection of oocytes and electrophysiological recordings were performed as follows. Physiological and pharmacological properties of NMDA receptor subunits were characterized in the X. laevis oocyte expression system. Full-length cDNAs encoding NMDA receptor subunits were linearized and used as templates for synthesis of cRNA by in vitro transcription using T3 or T7 RNA polymerase (Ambion, Inc., Austin, Tex.). The DNA template was removed by DNase I digestion, and the RNA was purified by phenol/chloroform extraction and ethanol precipitation. The RNA was resuspended in water at 1 μg/μl and stored in −80° C. freezer until use. X. laevis oocytes were harvested and defolliculated by digestion with collagenase (2 mg/ml) for 2 hrs. Twenty to 40 ng of cRNA were injected into each oocyte using glass pipettes with a tip size of 20-40 μm in diameter. Electrophysiological recordings were performed from whole oocytes 2-7 days after injection using a dual-electrode voltage-clamp amplifier (Model OC-725A, Warner Instrument, Hamden, Conn.). Micropipettes were filled with 3M KCl. The bath solution contained 115 mM NaCl, 2.5 mM KCl, 1.5 mM BaCl₂, and 10 mM Hepes at pH 7.4. Drugs were applied to oocytes via a rapid perfusion system. Data were collected at 2.2 to 11.1 Hz at room temperature using the MacLab/4e A/D converter (AD Instruments, Mountain View, Calif.). Data analysis was performed using the programs Chart (AD Instruments, Mountain View, Calif.) and Microsoft Excel.

Additionally, using the patch-clamp technique, outside-out patches were pulled from oocytes previously injected with NMDAR cRNAs. This method allowed the recording of NMDA-evoked or glycine-evoked single-channel currents, as described previously (Das et al., supra (1998)).

Several novel properties of the NR3B subunit were identified by functional expression of the B4 form in Xenopus oocytes. First, functional receptors (defined by the presence of glycine-evoked current) were assembled in oocytes after co-injection of cRNAs for the NR3B and NR1 subunits. Functional receptors were also observed after co-injection of NR3B cRNA with both NR1 and NR2A subunit cRNAs. However, oocytes expressing NR1/NR2A/NR3B receptors exhibited ligand-evoked currents that were 5 to 10 times larger than those expressing NR1/NR3B receptors. In contrast, no functional receptors were detected when NR3B cRNA was co-injected with either NR2A or NR3A alone in the absence of NR1. These results suggest that assembly of the NR3R subunit into a functional receptor complex requires association with the NR1 subunit.

Strikingly, unlike the previously published NMDARs, it was found that the NR1/NR3B receptor was activated by glycine alone, in the absence of glutamate or NMDA (FIG. 3a). D-serine, D-alanine, D-cycloserine and ACPC, all co-agonists of traditional NMDARs via activation of the glycine site on the NR1 subunit, each inhibited glycine-induced currents of NR1/NR3B receptors (FIGS. 3b1, b2, and b3). Importantly, NMDA, the traditional agonist for which this subclass of receptor was originally named, displayed little or no effect by itself or on the glycine-induced current of NR1/NR3B receptors (FIG. 3c). Aspartate and cysteine, previously shown to activate the glutamate binding site of NMDARs (containing NR2 subunits), slightly potentiated glycine-evoked currents of NR1/NR3B receptors, as did kainate which is known to activate non-NMDA (GluR5-7 and KA1,KA2) receptors (FIGS. 3d1, 3d2). Of note, glycine alone activated NR1/NR3B receptors with high efficacy despite the fact that by itself glycine fails to excite the previously studied NMDARs or any other type of glutamate receptors. For NR1/NR3B receptors, the EC₅₀ for glycine was estimated to be approximately 5 μM. A more precise calculation of EC₅₀ was precluded by the desensitization that occurs at high glycine concentrations (FIG. 3a).

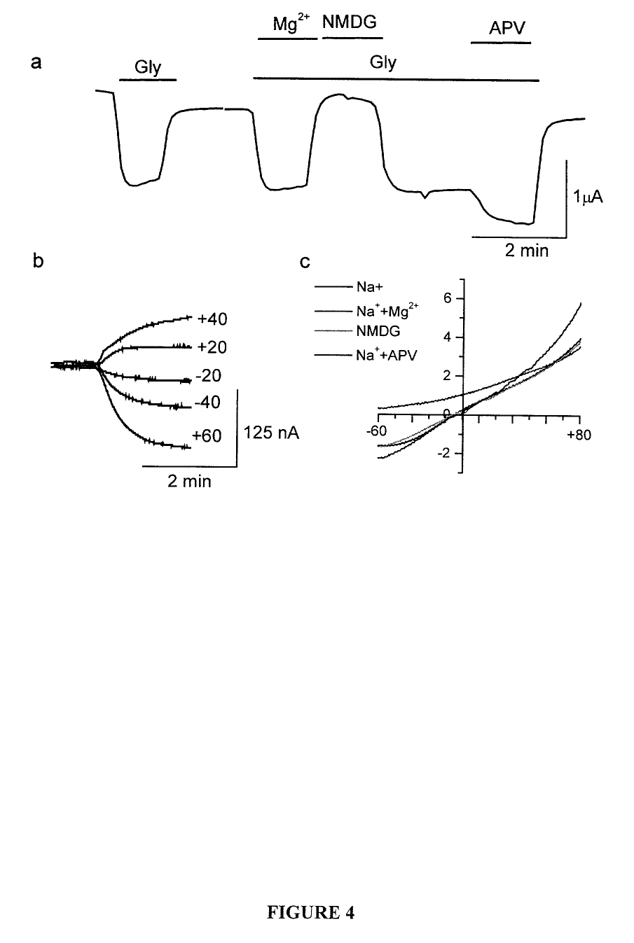

The altered ligand selectivity conferred by the NR3B subunit was demonstrated further by the totally unexpected effect of some NMDAR antagonists. For example, 2-amino-5-phosphonovalerate (APV), which normally acts as a competitive antagonist at the glutamate binding site of the known NMDARs, e.g., on NR2A in NR1/NR2A receptors, had little or no effect at 10 μM on glycine (10 μM)-evoked currents in NR1/NR3B receptors (FIG. 3f1); at higher concentrations (40 μM), APV actually potentiated glycine-evoked current somewhat (right-hand panel, FIG. 4a). In contrast, 5,7-dichlorokynurenate, a competitive antagonist of the NR1 subunit glycine binding site, completely abolished glycine-evoked currents of NR1/NR3B receptors. As expected, strychnine, an antagonist of the inhibitory, chloride permeable glycine receptor, had no effect (FIG. 3f2). Properties linked to the channel region were also altered by the presence of the NR3B subunit. Compared to NR1/NR2A receptors, NR1/NR3B receptors were drastically less sensitive to channel blockers such as Mg²⁺, MK-801, and memantine (FIGS. 3e1, e2, and e3).

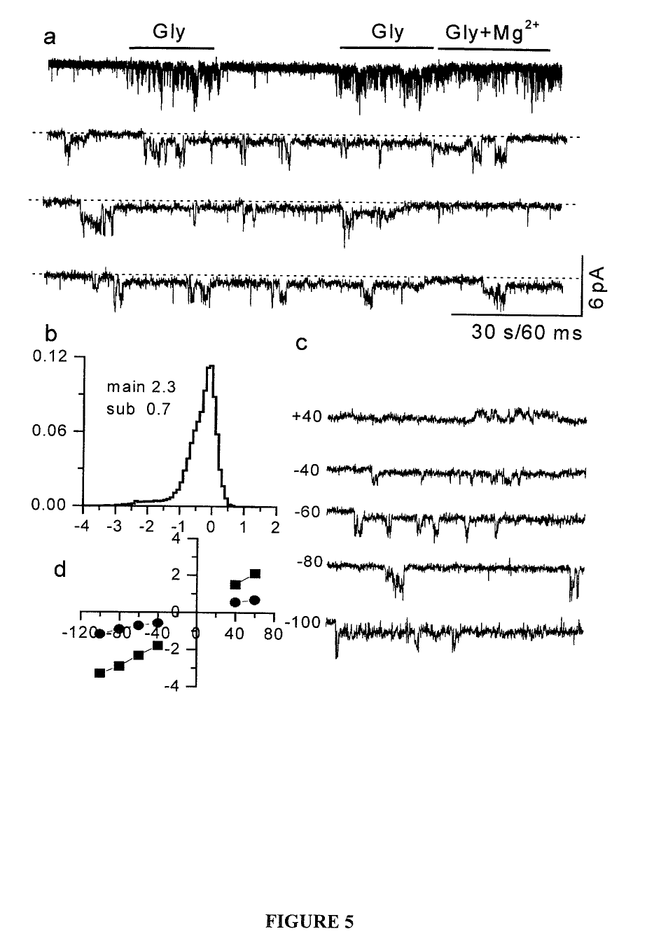

Like the more traditional NMDARs, it was found that NR1/NR3B receptor-operated channels were selectively permeable to cations. Replacement of all cations with N-methyl-D-gluconate (NMDG) in the external buffer completely abolished the glycine-induced inward current (FIG. 4a). Furthermore, replacement of all cations with Ca²⁺ also eliminated the inward current, and, unlike NR1/NR2 receptors, activation of NR1/NR3B receptors did not evoke a Ca²⁺-triggered Cl⁻ current when recorded in Ca²⁺-containing medium from Xenopus oocytes (data not shown). These results suggest that NR1/NR3B receptors display far less permeability to Ca²⁺ than NMDAR containing NR1 and NR2A-D subunits. Single-channel recording of NR1/NR3B receptors confirmed many of the aforementioned unique properties of these receptors, including activation by glycine alone and reduced sensitivity to Mg²⁺ (FIG. 5a). Recorded from outside-out patches of oocyte membrane, NR1/NR3B receptor-operated channels manifest a unitary conductance of ˜38 pS and a subconductance of 12 pS in the presence of 2 mM external Ba²⁺ (FIGS. 5a, c). When all anions in the patch electrode were replaced by impermeant gluconate, the conductance states remained unaffected at both positive and negative holding potentials (FIG. 5c), confirming that the channels were permeable to cations.

### EXAMPLE IV

This example shows characterization of biological activities of NR3A-containing excitatory glycine receptors.

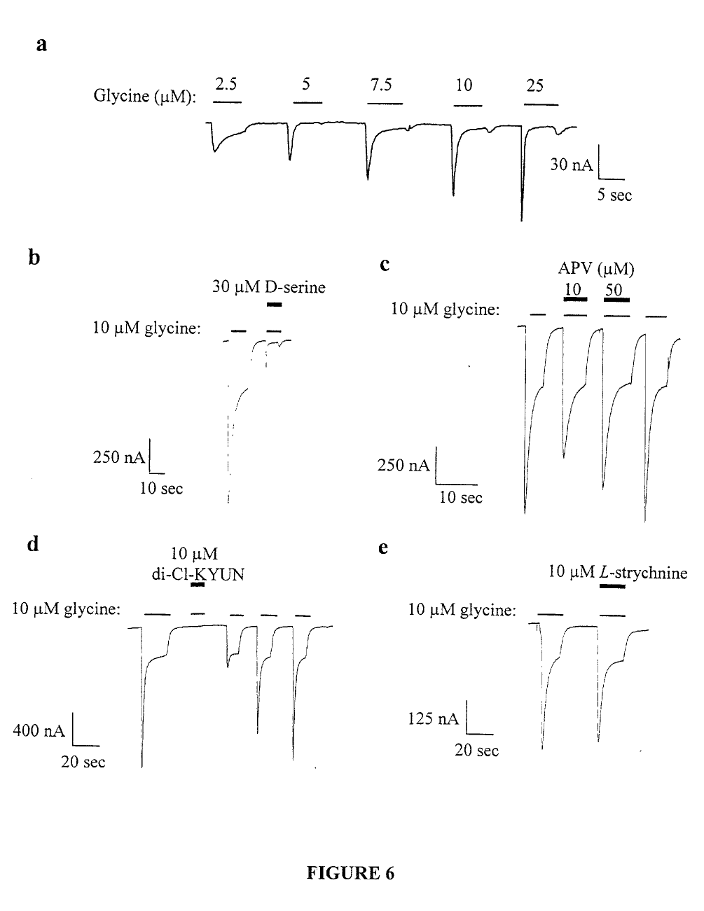

Because of the high degree of sequence similarity between NR3B and NR3A subunits, the ability of the NR3A subunit to form functional channels when co-expressed with NR1 was re-examined. Reminiscent of NR1/NR3B receptors, it was found that under specific conditions during two-electrode voltage recordings from oocytes, NR1/NR3A receptors could be activated by glycine alone (FIG. 6a). However, rapid desensitization of the current from NR1/NR3A receptors at micromolar concentrations of ligand rendered the observation of glycine-evoked currents extremely difficult, explaining why they had not been reported previously (Ciabarra et al., J. Neurosci. 15:6498-6508 (1995); Sucher et al., J. Neuroscience 15:6509-6520 (1995); Das et al., Nature 393:377-381 (1998).

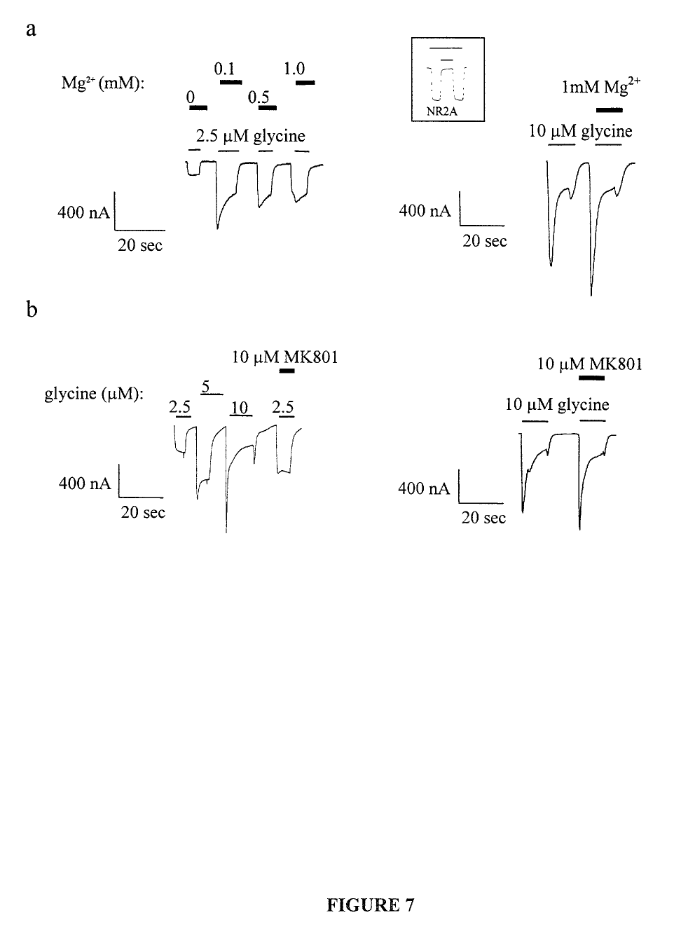

Similar to NR1/NR3B receptors, glycine-evoked currents of NR1/NR3A receptors were effectively blocked by D-serine (FIG. 6b). APV blocked the current only slightly (FIG. 6c). As in the case of NR1/NR3B receptors, glycine-evoked currents from NR1/NR3A receptors were blocked by 5,7-dichlorokynurenate (FIG. 6d), but not by strychnine (FIG. 6e). Interestingly, channel blockers, such as Mg²⁺ and MK-801, not only failed to block glycine-evoked currents of NR1/NR3A receptors, but actually potentiated these currents when low concentrations of glycine were used (FIGS. 7a, b). These findings suggest that NR3A-containing receptors, similar to their NR3B counterparts, manifest novel pharmacological and physiological properties. Additionally, NR1/NR3A receptors desensitize rapidly, distinguishing them from NR1/NR3B receptors.

### EXAMPLE V

This example shows the production of NR3B-specific antibodies using NR3B peptides.

In order to produce NR3B-specific antibodies, the XhoI-AvrII and MluI-XbaI fragments of rat NR3B cDNA (B4 form), which encode amino acids 89-238 and 927-1002, respectively, were subcloned into pET28 to facilitate expression of 6xHis-tagged NR3B fragments in bacteria. These regions of NR3B exhibit relatively poor homology with NR3A and other NMDA receptor subunits. The 6xHis-tagged proteins were expressed in BL21(DE3) cells and purified by metal affinity chromatography (Talon Purification Kit, Clontech, Palo Alto, Calif.). The purified proteins are used to immunize chickens (Aves Labs, Tigard, Oreg.) and rabbits (Covance, Princton, N.J.) for the production of anti-NR3B polyclonal antibodies.

All journal article, reference, sequence and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention.

What is claimed is:

1. An isolated NR3B nucleic acid molecule, comprising a nucleotide sequence which encodes a NR3B polypeptide having at least 80% identity to the amino acid sequence designated SEQ ID NO:6, wherein said polypeptide does not consist of the amino acid sequence designated SEQ ID NO:10 or 12.

2. The isolated nucleic acid molecule of claim 1, wherein said NR3B polypeptide comprises the amino acid sequence designated SEQ ID NO:6.

3. The isolated nucleic acid molecule of claim 1, wherein said NR3B polypeptide comprises the amino acid sequence designated SEQ ID NO:4.

4. The isolated nucleic acid molecule of claim 1, wherein said NR3B polypeptide comprises the amino acid sequence designated SEQ ID NO:2.

5. The isolated nucleic acid molecule of claim 1, wherein said NR3B polypeptide comprises the amino acid sequence designated SEQ ID NO:8.

6. The isolated nucleic acid molecule of claim 1, operatively linked to a promoter of gene expression.

7. A vector comprising the isolated nucleic acid molecule of claim 1.

8. A cell comprising the isolated nucleic acid molecule of claim 1.

9. The cell of claim 8, wherein said NR3B polypeptide is expressed at the cell membrane.

10. The cell of claim 8, wherein an NR1 polypeptide is further expressed at the cell membrane.

11. The cell of claim 10, wherein said NR1 polypeptide comprises a human, rat or mouse NR1 amino acid sequence.

12. The cell of claim 10, wherein said NR1 polypeptide comprises the amino acid sequence designated SEQ ID NO:14.

13. A method of producing an NR3B polypeptide, comprising expressing the nucleic acid molecule of claim 1 in vitro or in a cell under conditions suitable for expression of said polypeptide.

14. An isolated NR3B nucleic acid molecule, comprising a nucleotide sequence which encodes a functional fragment of an NR3B polypeptide, wherein said NR3B polypeptide comprises an amino acid sequence designated SEQ ID NO:2, 4, 6 or 8, wherein said functional fragment does not consist of the amino acid sequence designated SEQ ID NO:10 or 12.

15. The isolated nucleic acid molecule of claim 14, wherein said functional fragment binds glycine.

16. An isolated NR3B polynucleotide, comprising at least 17 contiguous nucleotides from the nucleotide sequence designated SEQ ID NO:1, 3, 5 or 7, or from the complement thereof, wherein said nucleic acid molecule does not consist of the nucleotide sequence designated SEQ ID NO:9, 11 or 13.

17. A method for detecting a nucleic acid molecule encoding a NR3B polypeptide in a sample, comprising contacting said sample with one or more polynucleotides according to claim 16, and detecting specific hybridization to said polynucleotide, thereby detecting a nucleic acid molecule encoding an NR3B polypeptide in said sample.

18. An isolated NR3B polypeptide, comprising an amino acid sequence having at least 80% identity to SEQ ID NO:6, wherein said polypeptide does not consist of the amino acid sequence designated SEQ ID NO:10 or 12.

19. The isolated polypeptide of claim 18, wherein said polypeptide comprises the amino acid sequence designated SEQ ID NO:6.

20. The isolated polypeptide of claim 18, wherein said polypeptide comprises the amino acid sequence designated SEQ ID NO:4.

21. The isolated polypeptide of claim 18, wherein said polypeptide comprises the amino acid sequence designated SEQ ID NO:2.

22. The isolated polypeptide of claim 18, wherein said polypeptide comprises the amino acid sequence designated SEQ ID NO:8.

23. The isolated polypeptide of claim 18, further comprising a membrane.

24. The isolated polypeptide of claim 18, which is associated with an NR1 polypeptide to form an excitatory glycine receptor.

25. The isolated polypeptide of claim 18, which is bound to glycine.

26. A functional fragment of an NR3B polypeptide, wherein said NR3B polypeptide comprises an amino acid sequence designated SEQ ID NO:2, 4, 6 or 8, wherein said functional fragment does not consist of the amino acid sequence designated SEQ ID NO:10 or 12.

27. The isolated polypeptide of claim 26, wherein said functional fragment binds glycine.

28. An isolated NR3B peptide, comprising at least 8 contiguous residues of the amino acid sequence designated SEQ ID NO:2, 4, 6 or 8, wherein said peptide does not consist of the amino acid sequence designated SEQ ID NO:10 or 12.

29. An isolated antibody or antigen binding fragment thereof which specifically binds the isolated NR3B polypeptide of claim 18.

30. A method of detecting an NR3B polypeptide in a sample, comprising contacting said sample with the antibody of claim 29, and detecting the presence of specific binding of said antibody to said sample, thereby detecting an NR3B polypeptide in said sample.

31. A method of detecting an NR3B ligand, comprising:
(a) contacting the polypeptide of claim 18 with one or more candidate compounds under conditions suitable for detecting binding to said polypeptide; and
(b) detecting a candidate compound that binds said polypeptide,
wherein said compound is characterized as an NR3B ligand.

32. A method of detecting an NR3B ligand, comprising:
(a) contacting the functional fragment of claim 27 with one or more candidate compounds under conditions suitable for detecting binding to said fragment; and
(b) detecting a candidate compound that binds said fragment,
wherein said compound is characterized as an NR3B ligand.

33. A composition, comprising an isolated excitatory glycine receptor.

34. The composition of claim 33, further comprising a cell membrane.

35. The composition of claim 34, wherein said cell is a Xenopus oocyte or mammalian cell.

36. The composition of claim 33, wherein said excitatory glycine receptor comprises an NR3B polypeptide and an NR1 polypeptide.

37. The composition of claim 36, wherein said excitatory glycine receptor further comprises an NR2 polypeptide.

38. The composition of claim 36, wherein said NR3B polypeptide comprises the amino acid sequence designated SEQ ID NO:2, 4, 6 or 8.

39. The composition of claim 36, wherein said NR1 polypeptide comprises a naturally-occurring human, rat or mouse NR1 amino acid sequence.

40. The composition of claim 33, wherein said excitatory glycine receptor comprises an NR3A polypeptide and an NR1 polypeptide.

41. The composition of claim 40, wherein said excitatory glycine receptor further comprises an NR2 polypeptide.

42. The composition of claim 40, wherein said NR3A polypeptide comprises a naturally-occurring human or rat NR3A amino acid sequence.

43. The composition of claim 40, wherein said NR1 polypeptide comprises a naturally-occurring human, rat or mouse NR1 amino acid sequence.

44. A method of detecting an excitatory glycine receptor ligand, comprising:
(a) contacting an excitatory glycine receptor with one or more candidate compounds under conditions suitable for detecting binding to said receptor; and
(b) detecting a candidate compound that binds said receptor,
wherein said compound is characterized as an excitatory glycine receptor ligand.

45. The method of claim 44, wherein said excitatory glycine receptor comprises an NR3B polypeptide and an NR1 polypeptide.

46. The method of claim 45, wherein said excitatory glycine receptor further comprises an NR2 polypeptide.

47. The method of claim 45, wherein said NR3B polypeptide comprises the amino acid sequence designated SEQ ID NO:2, 4, 6 or 8.

48. The method of claim 45, wherein said NR1 polypeptide comprises a naturally-occurring human, rat or mouse NR1 amino acid sequence.

49. The method of claim 44, wherein said excitatory glycine receptor comprises an NR3A polypeptide and an NR1 polypeptide.

50. The method of claim 49, wherein said excitatory glycine receptor further comprises an NR2 polypeptide.

51. The method of claim 49, wherein said NR3A polypeptide comprises a naturally-occurring human or rat NR3A amino acid sequence.

52. The method of claim 49, wherein said NR1 polypeptide comprises a naturally-occurring human, rat or mouse NR1 amino acid sequence.

53. The method of claim 44, wherein said contacting occurs in the presence of glycine.

54. A method of detecting an excitatory glycine receptor agonist or antagonist, comprising:
(a) contacting an excitatory glycine receptor with one or more candidate compounds under conditions suitable for detecting receptor activation; and
(b) detecting a candidate compound that alters said receptor activation,
wherein said compound is characterized as an excitatory glycine receptor agonist or antagonist.

55. The method of claim 54, wherein said excitatory glycine receptor comprises an NR3B polypeptide and an NR1 polypeptide.

56. The method of claim 55, wherein said excitatory glycine receptor further comprises an NR2 polypeptide.

57. The method of claim 55, wherein said NR3B polypeptide comprises the amino acid sequence designated SEQ ID NO:2, 4, 6 or 8.

58. The method of claim 55, wherein said NR1 polypeptide comprises a naturally-occurring human, rat or mouse NR1 amino acid sequence.

59. The method of claim 54, wherein said excitatory glycine receptor comprises an NR3A polypeptide and an NR1 polypeptide.

60. The method of claim 59, wherein said excitatory glycine receptor further comprises an NR2 polypeptide.

61. The method of claim 59, wherein said NR3A polypeptide comprises a naturally-occurring human or rat NR3A amino acid sequence.

62. The method of claim 59, wherein said NR1 polypeptide comprises a naturally-occurring human, rat or mouse NR1 amino acid sequence.

63. The method of claim 54, wherein said receptor activation is detected by assaying whole-cell currents.

64. The method of claim 54, wherein said receptor activation is detected by assaying single-channel currents.

65. The method of claim 54, wherein said receptor activation is detected by assaying ion fluxes using ion indicators.

66. The method of claim 63, wherein said cell is a Xenopus oocyte or mammalian cell that expresses said excitatory glycine receptor.

67. The method of claim 54, wherein said contacting occurs in the presence of glycine.

68. A method of modulating a cellular response to glycine or glutamate, comprising:
(a) introducing the nucleic acid molecule of claim 1 into a cell; and
(b) expressing the NR3B polypeptide encoded by said nucleic acid molecule in said cell,
whereby expression of said polypeptide modulates a cellular response to glycine or glutamate.

69. A method of modulating a cellular response to glycine or glutamate, comprising:
(a) introducing the nucleic acid molecule of claim 1 into a cell; and
(b) expressing the NR3B functional fragment encoded by said nucleic acid molecule in said cell,
whereby expression of said functional fragment modulates a cellular response to glycine or glutamate.

70. A method of modulating a cellular response to glycine or glutamate, comprising introducing an antisense nucleic acid molecule, a ribozyme molecule or a small interfering RNA (siRNA) molecule into said cell, wherein said molecule hybridizes to any of SEQ ID NO:1, 3, 5 or 7 and prevents translation of the encoded NR3B polypeptide.
